(19) United States
(12) Patent Application Publication
(10) Pub. No.: US 2023/0365944 A1
(43) Pub. Date: Nov. 16, 2023

(54) ENGINEERED STABLE LACTATE OXIDOREDUCTASES, COMPOSITIONS, DEVICES, KITS AND USES THEREOF

(71) Applicants: The University of North Carolina at Chapel Hill, Chapel Hill, NC (US); National University Corporation Tokyo University of Agriculture and Technology, Tokyo (JP)

(72) Inventors: Koji Sode, Chapel Hill, NC (US); Wakako Tsugawa, Tokyo (JP); Kentaro Hiraka, Tokyo (JP)

(21) Appl. No.: 18/029,224

(22) PCT Filed: Sep. 30, 2021
(86) PCT No.: PCT/US21/52938

§ 371 Date: Mar. 29, 2023

Related U.S. Application Data
(60) Provisional application No. 63/085,699, filed on Sep. 30, 2020.

(51) Int. Cl. C12N 9/04; C12Q 1/26; C12Q 1/00; C07K 14/80

(52) U.S. Cl. CPC C12N 9/0006; C12Q 1/26; C12Q 1/005; C07K 14/80; C07K 2319/00

(57) ABSTRACT

Compositions, devices, kits and methods are disclosed for assaying lactate with an engineered lactate oxidoreductase. The engineered lactate oxidoreductase has increased stability.

### CROSS-REFERENCE TO RELATED APPLICATION

This application claims the benefit of and priority to U.S. Provisional Application No. 63/085,699, filed Sep. 30, 2020, which is incorporated by reference herein in its entirety.

### FEDERALLY SPONSORED RESEARCH OR DEVELOPMENT

This invention was made with government support under grant number EEC-1160483 awarded by the National Science Foundation. The government has certain rights in the invention.

### REFERENCE TO A SEQUENCE LISTING SUBMITTED AS A TEXT FILE VIA EFS WEB

The Sequence Listing written in file 565131.txt is 86 kilobytes, was created on Sep. 29, 2021, and is hereby incorporated by reference.

### BACKGROUND

L-lactate is an important biomarker for clinical diagnostics, fitness monitoring in athletes, and food quality control. The L-lactate concentration can reflect lactic acidosis which is caused by tissue hypoxia or other underlying diseases such as liver disease or sepsis. In addition, monitoring the L-lactate concentration can indicate the lactate thresholds of athletes which is an indicator of endurance.

Current commercially available lactate oxidoreductases are not stable enough to be used as enzymes for long term continuous operation lactate monitoring, such as continuous interstitial fluid lactate monitoring and sweat lactate monitoring.

There remains a need to develop a stable lactate oxidoreductase that is suitable for lactate biosensing and continuous monitoring.

### BRIEF SUMMARY

Compositions, devices, kits, and methods are provided for assaying lactate and other biomolecules in a sample from a subject.

One embodiment is an engineered lactate oxidoreductase with increased stability as compared to the wild-type.

A further embodiment is an engineered lactate oxidoreductase, comprising a sequence having at least 90% sequence identity to any one of SEQ ID NOs:1-16, provided at least one of the amino acids at a position in said sequence corresponding to positions 10 to 30, positions 119-139, positions 154-174, or positions 175 to 195 of SEQ ID NO:1 is different from the amino acid occupying the corresponding position in said SEQ ID NO: 1-16.

Another embodiment is an engineered lactate oxidoreductase, comprising a sequence having at least 90% sequence identity to SEQ ID NO:1 provided at least one of positions 10 to 30 or positions 175 to 195 is different from the amino acid occupying the corresponding position in SEQ ID NO: 1.

Another embodiment is an engineered lactate oxidoreductase, comprising a modification at one or more amino acid positions selected from: (a) a position corresponding to position 20 of the amino acid sequence set forth in SEQ ID NO: 1, (b) a position corresponding to position 185 of the amino acid sequence set forth in SEQ ID NO: 1, (c) a position corresponding to position 129 of the amino acid sequence set forth in SEQ ID NO: 1, and (d) a position corresponding to position 164 of the amino acid sequence set forth in SEQ ID NO: 1.

Another embodiment is an engineered lactate oxidoreductase, comprising a modification at one or more amino acid positions selected from: (a) a position corresponding to position 20 of the amino acid sequence set forth in SEQ ID NO: 1 and (b) a position corresponding to position 185 of the amino acid sequence set forth in SEQ ID NO: 1.

Another embodiment is an engineered lactate oxidoreductase, comprising a sequence having at least 90% sequence identity to SEQ ID NO:1 provided at least one of position 20, position 129, position 164, or position 185 is different from the amino acid occupying the corresponding position in SEQ ID NO: 1.

Another embodiment is an engineered lactate oxidoreductase, comprising a sequence having at least 90% sequence identity to SEQ ID NO:1 provided at least one of positions 20 or positions 185 is different from the amino acid occupying the corresponding position in SEQ ID NO: 1.

Another embodiment is an engineered lactate oxidoreductase further comprising a modification at a position corresponding to position 96 of the amino acid sequence set forth in SEQ ID NO: 1, wherein the modification includes a substitution of the wild-type amino acid residue with an amino acid residue Leu. In another embodiment, the engineered lactate oxidoreductase further comprises a modification at a position corresponding to position 212 of the amino acid sequence set forth in SEQ ID NO: 1, wherein the modification includes a substitution of the wild-type amino acid residue with an amino acid residue Lys. In an embodiment, the wild-type amino acid residue at position 96 is Ala. In an embodiment, the wild-type amino acid residue at position 212 is Asn.

A further embodiment is an engineered lactate oxidoreductase further comprising a modification at a position corresponding to position 95 of the amino acid sequence set forth in SEQ ID NO: 1, wherein the modification includes a substitution of the wild-type amino acid residue with an amino acid residue Ser. In an embodiment, the wild-type amino acid residue at position 95 is Ala.

An embodiment is an engineered lactate oxidoreductase which includes a reduced oxidase activity as compared to the wild-type lactate oxidoreductase, an increased dehydrogenase activity compared to the wild-type lactate oxidoreductase, and/or an increased Km.

Another embodiment is an engineered lactate oxidoreductase comprising a fused cytochrome domain of flavocytochrome b2.

A further embodiment is said engineered lactate oxidoreductase comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, iii) a position corresponding to position 96 of the amino acid sequence set forth in SEQ ID NO: 1, wherein the modification includes a substitution of the wild-type amino acid residue with an amino acid residue Leu, iv) a position corresponding to position 212 of the amino acid sequence set forth in SEQ ID NO: 1, wherein the modification includes a substitution of the wild-type amino acid residue with an amino acid residue Lys, and v) a position corresponding to position 95 of the amino acid sequence set forth in SEQ ID NO: 1, wherein the modification includes a substitution of the wild-type amino acid residue with an amino acid residue Ser, and fused to a cytochrome domain of flavocytochrome b2. In an embodiment, the wild-type amino acid residue at position 20 is Val, the wild-type amino acid residue at position 185 is Val, the wild-type amino acid residue at position 96 is Ala, the wild-type amino acid residue at position 212 is Asn, and the wild-type amino acid residue at position 95 is Ala.

Another embodiment is an engineered lactate oxidoreductase comprising a modification at i) a position corresponding to position 129 of the amino acid sequence set forth in SEQ ID NO: 1, wherein the modification includes a substitution of the wild-type amino acid residue with an amino acid residue Cys, ii) a position corresponding to position 164 of the amino acid sequence set forth in SEQ ID NO: 1, wherein the modification includes a substitution of the wild-type amino acid residue with an amino acid residue Cys, iii) a position corresponding to position 96 of the amino acid sequence set forth in SEQ ID NO: 1, wherein the modification includes a substitution of the wild-type amino acid residue with an amino acid residue Leu, iv) a position corresponding to position 212 of the amino acid sequence set forth in SEQ ID NO: 1, wherein the modification includes a substitution of the wild-type amino acid residue with an amino acid residue Lys, and v) a position corresponding to position 95 of the amino acid sequence set forth in SEQ ID NO: 1, wherein the modification includes a substitution of the wild-type amino acid residue with an amino acid residue Ser, and fused to a cytochrome domain of flavocytochrome b2.

A further embodiment is said engineered lactate oxidoreductase comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, iii) a position corresponding to position 129 of the amino acid sequence set forth in SEQ ID NO: 1, wherein the modification includes a substitution of the wild-type amino acid residue with an amino acid residue Cys, iv) a position corresponding to position 164 of the amino acid sequence set forth in SEQ ID NO: 1, wherein the modification includes a substitution of the wild-type amino acid residue with an amino acid residue Cys, v) a position corresponding to position 96 of the amino acid sequence set forth in SEQ ID NO: 1, wherein the modification includes a substitution of the wild-type amino acid residue with an amino acid residue Leu, vi) a position corresponding to position 212 of the amino acid sequence set forth in SEQ ID NO: 1, wherein the modification includes a substitution of the wild-type amino acid residue with an amino acid residue Lys, and vii) a position corresponding to position 95 of the amino acid sequence set forth in SEQ ID NO: 1, wherein the modification includes a substitution of the wild-type amino acid residue with an amino acid residue Ser, and fused to a cytochrome domain of flavocytochrome b2.

### BRIEF DESCRIPTION OF THE SEVERAL VIEWS OF THE DRAWING(S)

Having thus described the subject matter in general terms, reference will now be made to the accompanying drawings, which are not necessarily drawn to scale.

FIG. 1 illustrates results for lactate oxidoreductase enzyme stability experiments.

FIG. 2 illustrates results from experiments with V20C and/or V185C single or double mutants (V20C/V185C; CC1) together with or without A96L.

FIG. 3 illustrates the oxidase activity assay.

FIG. 4 illustrates the dehydrogenase activity assay.

FIG. 5 illustrates results from gel filtration chromatography.

FIG. 6 illustrates native PAGE/TOF-MS analysis.

FIG. 7 illustrates results from activity assay and stability test.

FIG. 8 illustrates results from activity assay of A96L versus A96L CC1 mutants.

FIG. 9 illustrates results from enzyme property analysis of A96L, N212K, and A96L/N212K mutants.

FIG. 10 illustrates exemplary electrode fabrication and SDS-PAGE results.

FIG. 11 illustrates absorption spectra change of the b2LOx A96L/N212K mutant in the presence of lactate.

FIG. 12 illustrates absorption spectra change of the b2LOx (FcbLOx)

A96L/N212K CC1, and b2LOx A96L/N212K CC1 mutants with carbon nanotube binding peptide (CNTBP) fused in its N-terminal region, in the presence of lactate.

FIG. 13 illustrates results of temperature stability.

FIG. 14 illustrates exemplary electrode fabrication.

FIG. 15 illustrates stability test results for PES modified CC1 (V20C/V185C) mutants.

FIG. 16 illustrates results from continuous lactate monitoring.

FIG. 17 illustrates a method of preparing b2LOX and b2LOx A95S electrodes.

FIG. 18 illustrates test results from b2LOX and b2Lox A95S (b2LOxS) electrodes without using outer membrane.

FIG. 19 illustrates test results from b2LOX and b2LOx A95S (b2LOxS) electrodes with outer membrane.

FIG. 20 illustrates test results for substrate inhibition of various A96L/N212K mutants.

FIG. 21 illustrates the effect of the A95S mutation on A96L/N212K and b2LOx substrate inhibition.

FIG. 22 illustrates stability and substrate specificity in AvLOx, b2LOx, and b2LOxS.

FIG. 23 illustrates heme b absorption spectra before and after lactate addition, for AvLOx and b2LOx, with and without an A95S mutation.

FIG. 24 illustrates a thin film electrode for simultaneous testing of lactate and glucose, along with test results.

FIG. 25 illustrates the correlation between measured currents and lactate/glucose concentrations.

FIG. 26 illustrates interferents testing and stability testing of BcGDH- and b2LOxS-based lactate and glucose monitoring in artificial sweat.

FIG. 27 illustrates thermal stability results for various A96L/N212K mutants, with either intra- or inter-subunit disulfide bonds.

FIGS. 28 and 29 illustrate further thermal activity and stability data for A96L/N212K mutants, optionally also comprising CC1, CC2, or CC3 mutations.

FIG. 30 illustrates substrate inhibition data for A96L/N212K mutants, optionally also comprising CC1, CC2, or CC3 mutations.

FIG. 31 illustrates the effect of the CC3 mutation on the thermal stability of b2LOx.

FIGS. 32A-32D illustrate the sequence alignment of various AvLOx mutant sequences and fusion proteins.

FIGS. 33A-33F illustrate the sequence alignment of FMN-dependent α-hydroxyacid oxidizing flavoproteins.

### DETAILED DESCRIPTION

As described herein, engineered lactate oxidoreductases that advantageously show stability over time and/or temperature thereby making them suitable for lactate biosensing and continuous lactate monitoring. As described herein, engineered lactate oxidoreductases were designed based on modeling experiments to elucidate the positions for mutation where the resulting engineered enzymes exhibited the desired stability. Additionally, as described herein, certain mutations surprisingly provide improved stability and performance in electrochemical biosensors.

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Overview

L-Lactate biosensors employing L-lactate oxidase (LOx) as a molecular recognition element have been studied since the first report of enzyme glucose sensors. LOx (EC: 1.1.3.15) is a member of the flavin mononucleotide (FMN)-dependent α-hydroxyacid oxidizing flavoprotein family. LOx is a homotetrameric enzyme, composed of 4 identical subunits with MW of 40 kDa. Each subunit harbors one FMN as its cofactor. This enzyme oxidizes L-lactate to pyruvate by FMN reduction in the reductive half-reaction and uses oxygen as a natural electron acceptor to reoxidize FMN and generate hydrogen peroxide in the oxidative half-reaction.

In embodiments, provided herein are amino acid sequences of engineered stable lactate oxidoreductases, such as lactate oxidases, lactate dehydrogenases, glycolate oxidases, long-chain hydroxy acid oxidases, mandelate oxidases, lactate monooxygenase, mandelate dehydrogenase, and flavocytochrome b2, which are composed of homotetrameric quaternary structure. In embodiments, provided herein are methods of making stable lactate oxidoreductases and their application for lactate monitoring, including but not limited, electrochemical lactate enzyme sensors. Shown herein, tetrameric lactate oxidoreductases can have increased stability compared to wild type by introducing specific mutations in these molecules. In embodiments, “stable” with respect to lactate oxidoreductase refers to retained oxidase activity over time and/or heat compared to wild-type. For example, provided herein are stabilized lactate oxidoreductases which retain more than 50% of their initial activity after 10 minutes incubation at 70° C. In embodiments, the stabilized lactate oxidoreductases retain more than 55%, or 60%, or 65%, or 70%, or 75%, or 80%, or 85%, or 90%, or 95%, or 96%, or 97%, or 98%, or 99%, or 99.5%, or 99.9% of their initial activity after 10 minutes incubation at 70° C. In embodiments, the stabilized lactate oxidoreductases retain 100% of their initial activity after 10 minutes incubation at 70° C. In embodiments, the stabilized lactate oxidoreductases retain more than 10%, or 15%, or 20%, or 25%, or 30%, or 35%, or 40%, or 45%, or 50%, or 55%, or 60%, or 65%, or 70% of their initial activity after 60 minutes incubation at 70° C. In contrast, wild type or other engineered lactate oxidoreductases without the mutations provided herein, such as those that possess only mutations to eliminate oxidase activity, for electron mediator modification, or for fusion with heme protein, are inactivated after 10 minutes of incubation at 70° C.

In embodiments, disclosed herein are engineered lactate oxidoreductases comprising amino acid residues that are substituted with Cys residues which results in the formation of inter-subunit and/or intra-subunit disulfide bonds in homo-tetrameric lactate oxidoreductases. In embodiments, disclosed herein are engineered lactate oxidoreductases comprising amino acid residues substituted with Cys residues which results in the formation of inter-subunit and/or intra-subunit disulfide bonds in tetrameric LOx or a mutant thereof or in fusion enzymes with a heme protein. In embodiments, disclosed herein are engineered lactate oxidoreductases comprising amino acid residues which are substituted with Cys residues to form inter-subunit and/or intra-subunit disulfide bonds in Aerococcus viridans derived tetrameric LOx (AvLOx) or a mutant thereof or in fusion enzymes with a heme protein.

In embodiments, provided herein are various AvLOx mutants, harboring Cys residue substitutions. Surprisingly, certain type of mutants exhibits increased thermal stability compared to wild type or another AvLOx mutants, such as Ala96Leu, Asn212Lys, Ala96Leu/Asn212Lys, or fusion AvLOx harboring heme b domain from Pichia pastoris derived flavocytochrome b2 (lactate dehydrogenase), or combinations thereof.

Fusion with the cytochrome domain of flavocytochrome b2 enables the engineered lactate oxidoreductase to transfer electrons directly to the electrode. In other words, fusion with the cytochrome domain of flavocytochrome b2 turns the lactate oxidoreductase from a non-direct electron transfer (non-DET) enzyme to a DET enzyme.

In embodiments, the AvLOx mutants disclosed herein may be fused at their N-terminus to the C-terminus of the cytochrome domain of flavocytochrome b2. In embodiments, the cytochrome domain of flavocytochrome b2 is derived from Pichia pastoris. In embodiments, the cytochrome domain of flavocytochrome b2 is derived from Saccharomyces cerevisiae, Hansenula anomala, or Hansenula polymorpha.

In embodiments, the fusion is at a position corresponding to position 93 of the amino acid sequence set forth in SEQ ID NO: 5.

Flavocytochrome b2 from Pichia pastoris
(SEQ ID NO: 5)
MNDDERKVTPEELAKHNTGTDCWVAINGKVYDLTEFLPQHPGGRNVILKR
AGKDASKVFNPIHPPDAISKFLPADKFVGVLDGELPEEEEVLTDAEIERQ
ERIANKPPLSSMFNVYDFEYVAQNILDEAAWAYYSSAADDEITLRENHFA
YHKVFFRPRILVDVTNIELETEMLGIKTSAPFYISATALAKLGHPEGEVG
IAKGAGRGDIIQMISTLASCSLDETVAAAKEGQSQWFQLYVNSDREVAYN
MIKHCEELGIKGIFVTVDAPSLGNREKDRRMKFTEDTDVDLSGDGKTEVN
RSNGAAAALSSFIDTAVTWKDIAEFKRRTNLPIVIKGIQRTEDVILAAEH
GVDGVVLSNHGGRQLDGAPPSLQVLAECMPVLRQRGLDKKLEVFVDGGIR
RGTDIMKALCLGAKGVGLGRPFLYANSAYGPDGVEKAIDILKNELIMNMR
LLGVTKISDLSPEFVDTRPLFGLTANDRLFNNNYLDIEFPKFKDE
Flavocytochrome b2 from Saccharomyces
cerevisiae
(SEQ ID NO: 6)
MNKQKISPAEVAKHNKPDDCWVVINGYVYDLTRFLPNHPGGQDVIKFNAG
KDVTAIFEPLHAPNVIDKYIAPEKKLGPLQGSMPPELVCPPYAPGETKED
IARKEQLKSLLPPLDNIINLYDFEYLASQTLTKQAWAYYSSGANDEVTHR
ENHNAYHRIFFKPKILVDVRKVDISTDMLGSHVDVPFYVSATALCKLGNP
LEGEKDVARGCGQGVTKVPQMISTLASCSPEEIIEAAPSDKQIQWYQLYV
NSDRKITDDLVKNVEKLGVKALFVTVDAPSLGQREKDMKLKFSNTKAGPK
AMKKTNVEESQGASRALSKFIDPSLTWKDIEELKKKTKLPIVIKGVQRTE
DVIKAAEIGVSGVVLSNHGGRQLDFSRAPIEVLAETMPILEQRNLKDKLE
VFVDGGVRRGTDVLKALCLGAKGVGLGRPFLYANSCYGRNGVEEAIEILR
DEIEMSMRLLGVTSIAELKPDLLDLSTLKARTVGVPNDVLYNEVYEGPTL
TEFEDA
Flavocytochrome b2 from Hansenula anomala
(SEQ ID NO: 7)
MKDIELTPEIVSQHNKKDDLWVVLNGQVYDLTDFLPNHPGGQKIIIRYAG
KDATKIFVPIHPPDTIEKFIPPEKHLGPLVGEFEQEEEELSDEEIDRLER
IERKPPLSQMINLHDFETIARQILPPPALAYYCSAADDEVTLRENHNAYH
RIFFNPKILIDVKDVDISTEFFGEKTSAPFYISATALAKLGHPEGEVAIA
KGAGREDVVQMISTLASCSFDEIADARIPGQQQWYQLYVNADRSITEKAV
RHAEERGMKGLFITVDAPSLGRREKDMKMKFEADSDVQGDDGDIDRSQGA
SRALSSFIDPSLSWKDIAFIKSITKMPIVIKGVQRKEDVLLAAEHGLQGV
VLSNHGGRQLDYTRAPVEVLAEVMPILKERGLDQKIDIFVDGGVRRGTDV
LKALCLGAKGVGLGRPFLYAMSSYGDKGVTKAIQLLKDEIEMNMRLLGVN
KIEELTPELLDTRSIHNRAVPVAKDYLYEQNYQRMSGAEFRPGIED
Flavocytochrome b2 from Hansenula polymorpha
(SEQ ID NO: 8)
MPKRIVPVDEFVKHNRPDDCWVAIRGQVYDMTEFLPQHPGGQSPIIRYSG
HDATELFEQLHPKGTIEKNLPKDKHLGQLDGPAPTLEVAEDEFEEERLEN
VANMPNVNEVMNLHDFEYIAKKILPKGAWAYYSSGADDEVSMRENHYAYQ
RIYFRPRVLVDVSKVDTSTTLLGTPTSVPFYVSATALAKLGHPDGECSIA
RGAGKEGVIQMISTLASNSLEEIAAARVPGATQWFQLYVNEDRNVAFEMV
KKAERLGIKAIFVTVDAPSLGNREKDARVKFEGESDVQKSNEVVRSQGAS
RALSSFIDTRLTWDDVIKIKQSTKLPVLIKGVQRLEDVVRAVDDGFDGVV
LSNHGGRQLDTAPPPVELLAEVVPELRRRNKLRPDFEIFIDGGVRRGTDI
LKALALGGQNVRVGVGLGRPFLYANSAYGENGVRKAIQLLKDELEMDMRL
LGVRNLRELDETFVDTRRLIGRDAPDELYNQLYSPLKTVKFRNE

In embodiments, a lactate oxidoreductase mutant is provided. In embodiments, the lactate oxidoreductase mutant can be simultaneously modified at two positions corresponding to position 20 and 185 of the AvLOx amino acid sequence, wherein the wild-type amino acids are substituted with Cys residues in the mutant.

In embodiments, an AvLOx mutant is provided. In embodiments, the AvLOx mutant can be simultaneously modified at two positions corresponding to position 20 and 185 of the AvLOx amino acid sequence, wherein Val20 and Val185 in wild-type AvLOx is substituted with Cys residues in the mutant.

In embodiments, a device is provided for assaying lactate in a sample, where the device includes a stabilized LOx as described herein and optionally an electron mediator. In some instances, an enzyme electrode is provided, where the enzyme electrode includes a stabilized LOx as described herein that is immobilized on the electrode. In other embodiments, an enzyme sensor is provided for assaying lactate, where the enzyme sensor includes an enzyme electrode as described herein as a working electrode. In another embodiment, a kit is provided for assaying lactate in a sample, where the kit includes a stabilized LOx as described herein and optionally an electron mediator.

Provided herein are engineered lactate oxidoreductases with drastically increased stability and their production and applications. In embodiments, the lactate oxidoreductases are selected from the group consisting of lactate oxidases, lactate dehydrogenase, glycolate oxidase, long-chain hydroxy acid oxidases, mandelate oxidases, lactate monooxygenases, mandelate dehydrogenase, and flavocytochrome b2. These engineered enzymes are useful as an enzyme in a lactate diagnostic kit, the biomolecular recognition element of optical and electrochemical biosensors such as for disposable lactate sensor and for the implantable or wearable sensors for continuous lactate monitoring system, and for lactate based continuous energy scavenging system, such as lactate enzyme fuel cells and their applications.

Provided herein are engineered LOx suitable for lactate biosensing. These engineered LOx are created by introducing mutations to decrease its oxidative half reaction using oxygen as electron acceptor but maintaining or increasing its reaction using artificial electron acceptors. Additionally, provided herein are engineered LOx that add quasi-direct electron transfer efficiency by introducing mutations where electron acceptors are directly modified on the surface of enzyme. Furthermore, provided here in are fusion enzymes comprising LOx and heme proteins to make this enzyme capable of direct electron transfer.

### Definitions

The term “subject” refers to a mammal (e.g., a human) in need of a lactate concentration analysis. The subject may include dogs, cats, pigs, cows, sheep, goats, horses, rats, mice, non-human mammals, and humans. The term “subject” does not necessarily exclude an individual that is healthy in all respects and does not have or show signs of elevated lactate.

As used herein, the term “physiological conditions” refers to the range of conditions of temperature, pH, and tonicity (or osmolality) normally encountered within tissues in the body of a living human.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

Compositions or methods “comprising” or “including” one or more recited elements may include other elements not specifically recited. For example, a composition that “comprises” or “includes” a protein may contain the protein alone or in combination with other ingredients.

Designation of a range of values includes all integers within or defining the range, and all subranges defined by integers within the range.

The singular forms of the articles “a,” “an,” and “the” include plural references unless the context clearly dictates otherwise. For example, the term “an antigen” or “at least one antigen” can include a plurality of antigens, including mixtures thereof.

Statistically significant means p≤0.05.

Other definitions are provided below.

### III. Compositions

### Engineered Lactate Oxidoreductases

In one embodiment, an isolated, engineered lactate oxidoreductase that exhibits increased stability when compared to a wild-type lactate oxidoreductase is provided. In embodiments, an engineered stable lactate oxidoreductase that has decreased oxidase (or Ox) activity when compared to a wild-type lactate oxidoreductase while substantially retaining dehydrogenase (or Dh) activity is provided. In another embodiment, the engineered stable lactate oxidoreductase further exhibits an increased Dh activity when compared to the wild-type lactate oxidoreductase. In embodiments, the Dh/Ox ratio is higher in an engineered stable lactate oxidoreductase mutant than wild-type lactate oxidoreductase.

As used herein, “isolated,” with respect to a polypeptide (and also a polynucleotide), means a molecule (e.g., polypeptide, protein or polynucleotide) isolated from its natural environment or prepared using synthetic methods such as those known to one of skill in the art. Complete purification is not required in either case. The molecules described herein can be isolated and purified from normally associated material in conventional ways, such that in the purified preparation the molecule is the predominant species in the preparation. At the very least, the degree of purification is such that extraneous material in the preparation does not interfere with use of the molecule in the manner disclosed herein. The molecule is at least about 85% pure; alternatively, at least about 90% pure, alternatively, at least about 95% pure; and alternatively, at least about 99% pure.

As used herein, “about” means within a statistically meaningful range of a value or values such as a stated concentration, length, molecular weight, pH, sequence identity, time frame, temperature or volume. Such a value or range can be within an order of magnitude, typically within 20%, more typically within 10%, and even more typically within 5% of a given value or range. The allowable variation encompassed by “about” will depend upon the particular system under study, and can be readily appreciated by one of skill in the art.

The term “wild type” refers to entities having a structure and/or activity as found in a normal (as contrasted with mutant, diseased, altered, or so forth) state or context. Wild type genes and polypeptides often exist in multiple different forms (e.g., alleles). A “wild type amino acid residue” at a given position refers to the amino acid present at a given position in a wild type polypeptide.

As used herein, “mutant,” when used in connection with a polypeptide or protein such as an enzyme, means a variant containing a substitution in one or more of the amino acid residues on the polypeptide or protein at the indicated position(s). Mutant also is used for a polynucleotide encoding such a mutant polypeptide or protein.

As used herein, “a position corresponding to” means the position of an amino acid residue in a query amino acid sequence that is aligned with the amino acid residue in a reference amino acid sequence using software such as AlignX of Vector NTI with default parameters (available from Invitrogen; see, Lu & Moriyama (2004) Brief Bioinform. 5:378-88). Thus, “amino acid (AA) residue at a position corresponding to the position Y of the amino acid sequence set forth in SEQ ID NO: X” means the AA residue in a query amino acid sequence that is aligned with AA Y of SEQ ID NO: X when the query amino acid sequence is aligned with SEQ ID NO: X using AlignX of Vector NTI with default parameters. It should be noted that the AA Y of SEQ ID NO: X itself is also encompassed by this term.

As used herein, “oxidase activity” or “Ox activity” means an enzymatic activity of the engineered lactate oxidoreductase to catalyze the oxidation of L-lactate to pyruvate by utilizing oxygen as an electron acceptor. The oxidase activity may be assayed by measuring the amount of generated hydrogen peroxide (H₂O₂) by any method known in the art such as, for example, by reagents for H₂O₂ detection such as 4AA/TODB/POD (4-aminoantipyrine/N,N-bis(4-sulfobutyl)-3-methylaniline disodium salt/horseradish peroxidase) or by a platinum (Pt) electrode. In the context of the relative or quantitative activity, the oxidase activity is specifically defined to be the mole amount of the substrate (lactate) oxidized per unit time measured by the amount of generated H₂O₂ at about 25° C. in 10 mM PPB, pH 7.0, 1.5 mM TODB, 2 U/ml horseradish peroxidase (POD), and 1.5 mM 4-aminoantipyrine (4AA). The formation of quinoneimine dye may be measured spectrophotometrically at 546 nm. This measurement, because it depends on oxygen, can be influenced by exposure to oxygen and the presence of dissolved oxygen.

As used herein, “dehydrogenase activity” or “Dh activity” means an enzymatic activity of the engineered lactate oxidoreductase to catalyze the oxidation of L-lactate to pyruvate by utilizing an electron mediator other than oxygen as an electron acceptor. This measurement, because it does not depend on oxygen, is less influenced by dissolved oxygen. The dehydrogenase activity may be assayed by measuring the amount of electron transferred to the mediator using, for example, mPMS/DCIP (1-methoxy-5-methylphenazinium methylsulfate/2,6-dichloroindophenol), cPES (trifluoro-acetate-1-(3-carboxy-propoxy)-5-ethyl-phenanzinium, NA BM31_1144 (N,N-bis-(hydroxyethyl)-3-methoxy-nitrosoaniline hydrochloride, NA BM31_1008 (N,N-bis-hydroxyethyl-4-nitrosoaniline) and N-N-4-dimethyl-nitrosoaniline. In the context of the relative or quantitative activity, the dehydrogenase activity is specifically defined to be the mole amount of the substrate (e.g., lactate) oxidized per unit time measured by the amount of electron transferred to the mediator at about 25° C. in 10 mM PPB (pH 7.0), 0.6 mM DCIP, and 6 mM methoxy PMS (mPMS).

It is therefore desired with respect to electrochemical biosensors to modulate the lactate oxidoreductase's activity towards the electron mediator and away from oxygen. In one embodiment, the engineered lactate oxidoreductase therefore has a reduced oxidase activity when compared to a wild-type lactate oxidoreductase, while substantially retaining the dehydrogenase activity. In another embodiment, the engineered lactate oxidoreductase can have an oxidase activity of about 50% or less when compared to the wild-type lactate oxidoreductase. In another embodiment, the engineered lactate oxidoreductase has an oxidase activity of about 40% or less, about 30% or less, about 20% or less, or about 15% or less when compared to the wild-type lactate oxidoreductase. In another embodiment, the engineered lactate oxidoreductase can have an oxidase activity of about 30% or less when compared to the wild-type lactate oxidoreductase.

In addition, the engineered lactate oxidoreductase can have a dehydrogenase activity of about 50% or more when compared to a wild-type lactate oxidoreductase. Alternatively, the engineered lactate oxidoreductase has a dehydrogenase activity of about 60% or more, about 70% or more, about 80% or more, about 90% or more, or about 100% or more when compared to the wild-type lactate oxidoreductase.

In the wild-type lactate oxidoreductase, the oxidase activity is about 10% to about 100% or more of the dehydrogenase activity. When dissolved oxygen is present in an assay system, electrons generated by oxidizing the substrate can be transferred to oxygen. Thus, the enzyme activity measured in the presence of an electron mediator will be greatly affected by the dissolved oxygen concentration. In certain embodiments, the engineered lactate oxidoreductase as described herein has a ratio of dehydrogenase/oxidase activity of about 3.0 or more, about 4.0 or more, about 5.0 or more, about 6.0 or more, about 7.0 or more, about 8.0 or more, about 10.0 or more, or about 15 or more. In one embodiment, the engineered lactate oxidoreductase as described herein has a ratio of dehydrogenase/oxidase activity of about 16.0. In certain embodiments, the engineered lactate oxidoreductase as described herein has a ratio of dehydrogenase/oxidase activity of from about 1.0 to about 30; or from about 5 to about 25; or from about 10 to about 20; or from about 14 to about 18.

In another embodiment, the engineered lactate oxidoreductase has a ratio of dehydrogenase/oxidase activity of about 1:1 or more, about 2:1 or more, about 3:1 or more, about 4:1 or more, about 5:1 or more, about 6:1 or more, about 7:1 or more, about 8:1 or more, about 9:1 or more, about 10:1 or more, about 11:1 or more, about 12:1 or more, or about 13:1 or more. In another embodiment, the engineered lactate oxidoreductase as described herein has a ratio of dehydrogenase/oxidase activity of from about 50% to about 15:1; or from about 1:1 to about 15:1; or from about 2:1 to about 15:1; or from about 3:1 to about 15:1; or from about 4:1 to about 15:1; or from about 5:1 to about 15:1; or from about 5:1 to about 14:1; or from about 5:1 to about 13:1; or from about 5:1 to about 12:1; or from about 5:1 to about 11:1; or from about 5:1 to about 10:1; or from about 6:1 to about 15:1; or from about 7:1 to about 15:1; or from about 8:1 to about 15:1; or from about 9:1 to about 15:1; or from about 10:1 to about 15:1. In another embodiment, the engineered lactate oxidoreductase has a ratio of dehydrogenase/oxidase activity of about 100% or more, about 200% or more, about 300% or more, about 400% or more, about 500% or more, about 600% or more, about 700% or more, about 800% or more, about 900% or more, about 1000% or more, about 1100% or more, about 1200% or more, about 1300% or more. In another embodiment, the engineered lactate oxidoreductase as described herein has a ratio of dehydrogenase/oxidase activity of from about 50% to about 1500%; or from about 100% to about 1500%; or from about 200% to about 1500%; or from about 300% to about 1500%; or from about 400% to about 1500%; or from about 500% to about 1500%; or from about 500% to about 1400%; or from about 500% to about 1300%; or from about 500% to about 1200%; or from about 500% to about 1100%; or from about 500% to about 1000%; or from about 600% to about 1500%; or from about 700% to about 1500%; or from about 800% to about 1500%; or from about 900% to about 1500%; or from about 1000% to about 1500%. In certain embodiments, since the dehydrogenase activity exceeds the oxidase activity, the enzyme activity of the engineered lactate oxidoreductase will be less affected by the dissolved oxygen concentration, which is advantageous in utilizing the engineered lactate oxidoreductase in a clinical diagnosis with a sample.

It should be understood that the numbering of the amino acid sequence for engineered lactate oxidoreductase herein begins at an initial Met and that the claimed engineered lactate oxidoreductase may or may not have the signal peptide. Examples of amino acid sequences for the engineered lactate oxidoreductase include, those having at least 90% sequence identity to any one of SEQ ID NOs:1-16, provided at least one of the amino acids at a position in said sequence corresponding to positions 10 to 30, positions 119 to 139, positions 154 to 174, or positions 175 to 195 of SEQ ID NO:1 is different from the amino acid occupying the corresponding position in said SEQ ID NOs: 1-16. Examples of amino acid sequences for the engineered lactate oxidoreductase also include those having at least 90% sequence identity to SEQ ID NO:1 provided at least one of positions 10 to 30 or positions 175 to 195 is different from the amino acid occupying the corresponding position in SEQ ID NO: 1.

In embodiments, provided herein is an engineered lactate oxidoreductase having at least 90% sequence identity to any one of SEQ ID NOs:1-16, provided at least one of positions 20, 129, 164, or 185 is different from the amino acid occupying the corresponding position in SEQ ID NO:1. In embodiments, provided herein is an engineered lactate oxidoreductase having at least 90% sequence identity to SEQ ID NO:1, provided at least one of positions 20, 129, 164, or 185 is different from the amino acid occupying the corresponding position in SEQ ID NO:1. In embodiments, provided herein is an engineered lactate oxidoreductase having at least 90% sequence identity to SEQ ID NO:1 provided at least one of positions 20 or 185 is different from the amino acid occupying the corresponding position in SEQ ID NO:1. In an embodiment, provided herein is an engineered lactate oxidoreductase modified in at least one position corresponding to 20 or 185 of SEQ ID NO: 1.

(SEQ ID NO: 1)
MNNNDIEYNAPSEIKYIDVVNTYDLEEEASKVVPHGGFNYIAGASGDEWT
KRANDRAWKHKLLYPRLAQDVEAPDTSTEILGHKIKAPFIMAPIAAHGLA
HTTKEAGTARAVSEFGTIMSISAYSGATFEEISEGLNGGPRWFQIYMAKD
DQQNRDILDEAKSDGATAIILTADSTVSGNRDRDVKNKFVYPFGMPIVQR
YLRGTAEGMSLNNIYGASKQKISPRDIEEIAAHSGLPVFVKGIQHPEDAD
MAIKAGASGIWVSNHGARQLYEAPGSFDTLPAIAERVNKRVPIVFDSGVR
RGEHVAKALASGADVVALGRPVLFGLALGGWQGAYSVLDYFQKDLTRVMQ
LTGSQNVEDLKGLDLFDNPYGYEY

In embodiments, an engineered lactate oxidoreductase is provided, comprising a modification at i) a position corresponding to position 20 of the amino acid sequence set forth in SEQ ID NO: 1, wherein the modification includes a substitution of the wild-type amino acid residue with an amino acid residue Cys or ii) a position corresponding to position 185 of the amino acid sequence set forth in SEQ ID NO: 1, wherein the modification includes a substitution of the wild-type amino acid residue with an amino acid residue Cys.

In embodiments, an engineered lactate oxidoreductase is provided, comprising i) a modification at a position corresponding to position 20 of the amino acid sequence set forth in SEQ ID NO: 1, wherein the modification includes a substitution of the amino acid residue Val with an amino acid residue Cys and ii) a position corresponding to position 185 of the amino acid sequence set forth in SEQ ID NO: 1, wherein the modification includes a substitution of the wild-type amino acid residue with an amino acid residue Cys.

In embodiments, an engineered lactate oxidoreductase is provided, comprising i) a modification at a position corresponding to position 20 of the amino acid sequence set forth in SEQ ID NO: 1, wherein the modification includes a substitution of the amino acid residue Val with an amino acid residue Cys or ii) a position corresponding to position 185 of the amino acid sequence set forth in SEQ ID NO: 1, wherein the modification includes a substitution of the amino acid residue Val with an amino acid residue Cys.

In embodiments, an engineered lactate oxidoreductase is provided, comprising a modification at i) a position corresponding to position 20 of the amino acid sequence set forth in SEQ ID NO: 1, wherein the modification includes a substitution of the amino acid residue Val with an amino acid residue Cys and ii) a position corresponding to position 185 of the amino acid sequence set forth in SEQ ID NO: 1, wherein the modification includes a substitution of the amino acid residue Val with an amino acid residue Cys.

In embodiments, the engineered lactate oxidoreductase is fused with heme or amine reactive phenazine ethosulfate (arPES).

In embodiments, the engineered lactate oxidoreductase provided herein comprises a modification at a position corresponding to position 95 of the amino acid sequence set forth in SEQ ID NO: 1, wherein the modification includes a substitution of the wild-type amino acid residue with an amino acid residue Ser.

In embodiments, the engineered lactate oxidoreductase provided herein comprises a modification at a position corresponding to position 95 of the amino acid sequence set forth in SEQ ID NO: 1, wherein the modification includes a substitution of the amino acid residue Ala with an amino acid residue Ser. The A95S mutant shows an increased Km.

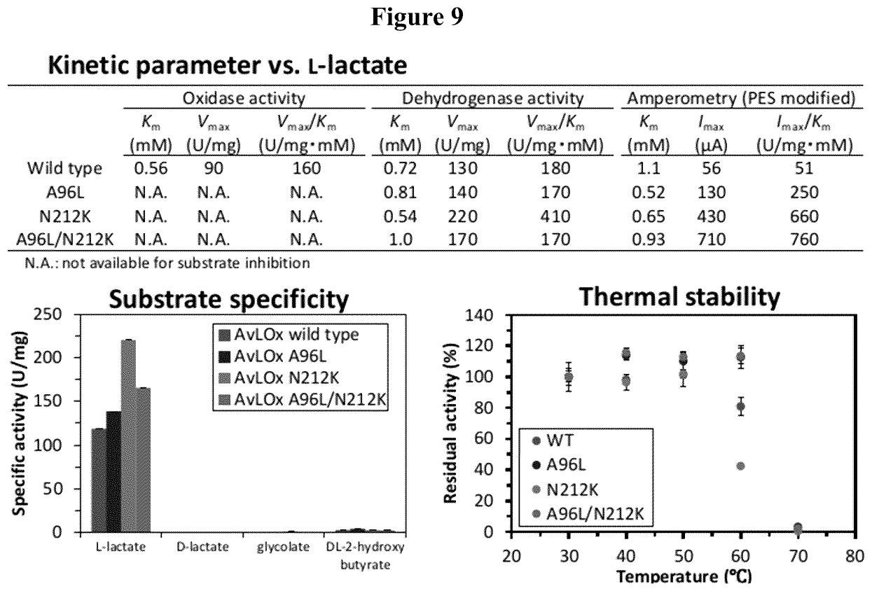

In embodiments, the engineered lactate oxidoreductase provided herein further comprises an A96L or N212K modification or both. An A96L/N212K lactate oxidoreductase mutant showed high catalytic activity/current, specificity and stability (FIG. 9).

In embodiments, the engineered lactate oxidoreductase provided herein comprises a modification at i) a position corresponding to position 129 of the amino acid sequence set forth in SEQ ID NO: 1, wherein the modification includes a substitution of the wild-type amino acid residue with an amino acid residue Cys, ii) a position corresponding to position 164 of the amino acid sequence set forth in SEQ ID NO: 1, wherein the modification includes a substitution of the wild-type amino acid residue with an amino acid residue Cys, and iii) a position corresponding to position 96 of the amino acid sequence set forth in SEQ ID NO: 1, wherein the modification includes a substitution of the wild-type amino acid residue with an amino acid residue Leu.

In embodiments, an engineered lactate oxidoreductase is provided, comprising a modification at i) a position corresponding to position 20 of the amino acid sequence set forth in SEQ ID NO: 1, wherein the modification includes a substitution of the amino acid residue Val with an amino acid residue Cys, ii) a position corresponding to position 185 of the amino acid sequence set forth in SEQ ID NO: 1, wherein the modification includes a substitution of the amino acid residue Val with an amino acid residue Cys, and iii) a position corresponding to position 96 of the amino acid sequence set forth in SEQ ID NO: 1, wherein the modification includes a substitution of the amino acid residue Ala with an amino acid residue Leu.

In another embodiment, the engineered lactate oxidoreductase provided herein comprises a modification at i) a position corresponding to position 129 of the amino acid sequence set forth in SEQ ID NO: 1, wherein the modification includes a substitution of the wild-type amino acid residue with an amino acid residue Cys, ii) a position corresponding to position 164 of the amino acid sequence set forth in SEQ ID NO: 1, wherein the modification includes a substitution of the wild-type amino acid residue with an amino acid residue Cys, and iii) a position corresponding to position 212 of the amino acid sequence set forth in SEQ ID NO: 1, wherein the modification includes a substitution of the wild-type amino acid residue with an amino acid residue Lys.

In another embodiment, an engineered lactate oxidoreductase is provided, comprising a modification at i) a position corresponding to position 20 of the amino acid sequence set forth in SEQ ID NO: 1, wherein the modification includes a substitution of the amino acid residue Val with an amino acid residue Cys, ii) a position corresponding to position 185 of the amino acid sequence set forth in SEQ ID NO: 1, wherein the modification includes a substitution of the amino acid residue Val with an amino acid residue Cys, and iii) a position corresponding to position 212 of the amino acid sequence set forth in SEQ ID NO: 1, wherein the modification includes a substitution of the amino acid residue Asn with an amino acid residue Lys.

In another embodiment, the engineered lactate oxidoreductase provided herein comprises a modification at i) a position corresponding to position 129 of the amino acid sequence set forth in SEQ ID NO: 1, wherein the modification includes a substitution of the wild-type amino acid residue with an amino acid residue Cys, ii) a position corresponding to position 164 of the amino acid sequence set forth in SEQ ID NO: 1, wherein the modification includes a substitution of the wild-type amino acid residue with an amino acid residue Cys, iii) a position corresponding to position 96 of the amino acid sequence set forth in SEQ ID NO: 1, wherein the modification includes a substitution of the wild-type amino acid residue with an amino acid residue Leu, and iv) a position corresponding to position 212 of the amino acid sequence set forth in SEQ ID NO: 1, wherein the modification includes a substitution of the wild-type amino acid residue with an amino acid residue Lys.

In another embodiment, an engineered lactate oxidoreductase is provided, comprising a modification at i) a position corresponding to position 20 of the amino acid sequence set forth in SEQ ID NO: 1, wherein the modification includes a substitution of the amino acid residue Val with an amino acid residue Cys, ii) a position corresponding to position 185 of the amino acid sequence set forth in SEQ ID NO: 1, wherein the modification includes a substitution of the amino acid residue Val with an amino acid residue Cys, iii) a position corresponding to position 96 of the amino acid sequence set forth in SEQ ID NO: 1, wherein the modification includes a substitution of the amino acid residue Ala with an amino acid residue Leu, and iv) a position corresponding to position 212 of the amino acid sequence set forth in SEQ ID NO: 1, wherein the modification includes a substitution of the amino acid residue Asn with an amino acid residue Lys.

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” refers to the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

### Engineered Lactate Oxidoreductase-Encoding Polynucleotides

In one embodiment, an isolated polynucleotide that encodes for an engineered lactate oxidoreductase as described herein.

The terms “nucleic acid” and “polynucleotide,” used interchangeably herein, refer to polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The polynucleotide encoding the wild-type lactate oxidoreductase may be cloned from the genome of respective organisms using PCR or other known techniques. Then, mutations may be introduced by techniques such as site-directed mutagenesis, PCR mutagenesis or any other known techniques. The amino acid residue to be mutated may be identified using any software for sequence alignment available in the art. Alternatively, polynucleotides coding for the for the engineered lactate oxidoreductase may be prepared by PCR using a series of chemically synthesized oligonucleotides, or fully synthesized. Examples of nucleotide sequences for the engineered lactate oxidoreductase can include, but are not limited to, those encoding an amino acid sequence as set forth in any one of SEQ ID NOS. 2-16 modified at least at one of a position corresponding to position 20, 129, 164, and 185 of SEQ ID NO: 1. Additional examples of nucleotide sequences for the engineered lactate oxidoreductase can include, but are not limited to, those encoding an amino acid sequence as set forth in any one of SEQ ID NOS. 2-16 modified at least at one of a position corresponding to position 20, 129, 164, and 185 of SEQ ID NO: 1 and further modified at least at one of a position corresponding to position 96 and 212 of SEQ ID NO: 1. Additional examples of nucleotide sequences for the engineered lactate oxidoreductase can include, but are not limited to, those encoding an amino acid sequence as set forth in any one of SEQ ID NOS. 2-16 modified at least at one of a position corresponding to position 20, 129, 164, and 185 of SEQ ID NO: 1 and further modified at least at one of a position corresponding to position 96 and 212 of SEQ ID NO: 1 and further modified at a position corresponding to position 95 of SEQ ID NO: 1.

In other embodiments, examples of nucleotide sequences for the engineered lactate oxidoreductase can include, but are not limited to, those encoding an amino acid sequence as set forth in any one of SEQ ID NOS. 2-4 and 9-10 modified at least at one of a position corresponding to position 20 and 185 of SEQ ID NO: 1. Additional examples of nucleotide sequences for the engineered lactate oxidoreductase can include, but are not limited to, those encoding an amino acid sequence as set forth in any one of SEQ ID NOS. 2-4 and 9-10 modified at least at one of a position corresponding to position 20 and 185 of SEQ ID NO: 1 and further modified at least at one of a position corresponding to position 96 and 212 of SEQ ID NO: 1. Additional examples of nucleotide sequences for the engineered lactate oxidoreductase can include, but are not limited to, those encoding an amino acid sequence as set forth in any one of SEQ ID NOS. 2-4 and 9-10 modified at least at one of a position corresponding to position 20 and 185 of SEQ ID NO: 1 and further modified at least at one of a position corresponding to position 96 and 212 of SEQ ID NO: 1 and further modified at a position corresponding to position 95 of SEQ ID NO: 1.

Lactate oxidase from Enterococcus hirae
NBRC 3181
(SEQ ID NO: 2)
MEKVYQAGTHEGMIDFINMEDLELAATQVIPSGGYGYISSGAGDLFTYRE
NQKAFNHQLVIPHVLKDVELPDTTTYFSDETLAAPIIMAPVAAHGLAHEQ
AEKASAKGVSEFGTIYTASSYASCTLEEIRAAGGPEAPQWFQFYMSKDDG
INLDILEMAKRNGAKAVVLTADATVGGNRETDRRNGFTFPLPMPIVQAYQ
SGVGQTMDAVYKSSKQKLSPKDIEFITTHSELPVYVKGVQSEDDVYRSLD
AGAQGIWVSNHGGRQLDGGPASFDSLRYVAEAVDKRVPIVFDSGVRRGQH
IFKAIASGADLVAIGRPAIYGLSLGGSTGIKQVFDFFKTELEMVMQLAGT
QTVEDIKNAKLRENRFMC
Lactate oxidase from Enterococcus spp
NBRC 3427
(SEQ ID NO: 3)
MEKTYQAGTNEGIVDFINMEDLEIAASQVIPAGGYGYISSGAGDLFTYQE
NERAFNHQLIIPHVLRDVELPDTTTHFDEETLTAPIIMAPVAAHGLAHVK
AEKASAKGVADFGTIYTASSYASCTLEEIREAGGEKAPQWFQFYMSKDNE
INLDILEVAKRNGAKAIVLTADATVGGNRETDRRNGFTFPLPMPIVQAYQ
SGVGQTMDAVYKSSKQKLSPKDVEFIAAHSDLPVYVKGVQSEEDVYRSLE
SGAGGIWVSNHGGRQLDGGPAAFDSLQYVADAVDKRVPIVFDSGVRRGQH
VFKAIASGADLVAIGRPVIYGLSLGGSTGVRQVFDFFKTELEMVMQLAGT
QTVEDIKKIKLRENRFI
Glycolate oxidase from Spinacia oleracea
(SEQ ID NO: 4)
MEITNVNEYEAIAKQKLPKMVYDYYASGAEDQWTLAENRNAFSRILFRPR
ILIDVTNIDMTTTILGFKISMPIMIAPTAMQKMAHPEGEYATARAASAAG
TIMTLSSWAT+NLSSVEEVASTGPGIRFFQLYVYKDRNVVAQLVRRAERA
GFKAIALTVDTPRLGRREADIKNRFVLPPFLTLKNFEGIDLGKMDKANDS
GLSSYVAGQIDRSLSWKDVAWLQTITSLPILVKGVITAEDARLAVQHGAA
GIIVSNHGARQLDYVPATIMALEEVVKAAQGRIPVFLDGGVRRGTDVFKA
LALGAAGVFIGRPVVFSLAAEGEAGVKKVLQMMRDEFELTMALSGCRSLK
EISRSHIAADWDGPSSRAVARL
Lactate dehydrogenase from Escherichia coli
(SEQ ID NO: 9)
MIISAASDYRAAAQRILPPFLFHYMDGGAYSEYTLRRNVEDLSEVALRQR
ILKNMSDLSLETTLFNEKLSMPVALAPVGLCGMYARRGEVQAAKAADAHG
IPFTLSTVSVCPIEEVAPAIKRPMWFQLYVLRDRGFMRNALERAKAAGCS
TLVFTVDMPTPGARYRDAHSGMSGPNAAMRRYLQAVTHPQWAWDVGLNGR
PHDLGNISAYLGKPTGLEDYIGWLGNNFDPSISWKDLEWIRDFWDGPMVI
KGILDPEDARDAVRFGADGIVVSNHGGRQLDGVLSSARALPAIADAVKGD
IAILADSGIRNGLDVVRMIALGADTVLLGRAFLYALATAGQAGVANLLNL
IEKEMKVAMTLTGAKSISEITQDSLVQGLGKELPAALAPMAKGNAA
Lactate dehydrogenase from Pseudomonas
stutzeri SDM
(SEQ ID NO: 10)
MIISASTDYRAAAKRKLPPFLFHYVDGGAYAEHTLRRNVEDLASIALRQR
VLRNMSELSLETQLFGETLSMPVALAPVGLTGMLARRGEVQAARAADKKG
IPFTLSTVARYRDAHSGMSGPNASVRRILQAMAHPLWAWDVGLHGKPHDL
GNITTYRGHTTGLEDYIGWLAANFDPSISWKDLEWIREFWDGPMVIKGIL
DAEDARDAVTFGADGIIVSNHGGRQLDGVMSSARAMPAIADAVKGDLKIL
ADSGIRNGLDVVRMIALGADTVLLGRAFAYALAVDGEAGVTNLLDLIEKE
MRVAMVLTGTKTISEISADSLVREVGHYQTATTSADV
Glycolate oxidase from Homo sapiens
(SEQ ID NO: 11)
MLPRLICINDYEQHAKSVLPKSIYDYYRSGANDEETLADNIAAFSRWKLY
PRMLRNVAETDLSTSVLGQRVSMPICVGATAMQRMAHVDGELATVRACQS
LGTGMMLSSWATSSIEEVAEAGPEALRWLQLYIYKDREVTKKLVRQAEKM
GYKAIFVTVDTPYLGNRLDDVRNRFKLPPQLRMKNFETSTLSFSPEENFG
DDSGLAAYVAKAIDPSISWEDIKWLRRLTSLPIVAKGILRGDDAREAVKH
GLNGILVSNHGARQLDGVPATIDVLPEIVEAVEGKVEVFLDGGVRKGTDV
LKALALGAKAVFVGRPIVWGLAFQGEKGVQDVLEILKEEFRLAMALSGCQ
NVKVIDKTLVRK
Long-chain hydroxy acid oxidase from
Rattus norvegicus
(SEQ ID NO: 12)
PLVCLADFKAHAQKQLSKTSWDFIEGEADDGITYSENIAAFKRIRLRPRY
LRDMSKVDTRTTIQGQEISAPICISPTAFHSIAWPDGEKSTARAAQEANI
CYVISSYASYSLEDIVAAAPEGFRWFQLYMKSDWDFNKQMVQRAEALGFK
ALVITIDTPVLGNRRRDKRNQLNLEANILLKDLRALKEEKPTQSVPVSFP
KASFCWNDLSLLQSITRLPIILKGILTKEDAELAMKHNVQGIVVSNHGGR
QLDEVSASIDALREVVAAVKGKIEVYMDGGVRTGTDVLKALALGARCIFL
GRPILWGLACKGEDGVKEVLDILTAELHRCMTLSGCQSVAEISPDLIQFS
RL
Mandelate oxidase from Amycolatopsis
orientalis
(SEQ ID NO: 13)
MTHLCLDDLERAARTVLPGEIWDFLAGGSGAEASLEANRAALERIFVIPR
MLRDLTGATGEAEVLGRPAAVPMAVAPVAYQRLFHPEGELAAARAARDAG
VPYTICTLSSVPLEEIAAVGGRPWFQLYWLRDEKRSLELVRRAEDAGCEA
IVFTVDVPWMGRRLRDLRNGFALPDSVTAANFDAGDAAHRRTRGQSAVAE
HTAREFAPATWESVEAVRAHTDLPVVLKGILAVEDATRAVDAGVGGIVVS
NHGGRQLDSAVPGIEMLGEIAAALSGWDGEVLLDGGIRSGGDILKALALG
ASAVLVGRPVMWGLAAGGEDGARQSLELLAVEFRNALGLAGCDSVSAARR
LGTRVLSR
Mandelate oxidase from Streptomyces
coelicolor
(SEQ ID NO: 14)
MREPLTLDDFARLARGQLPAATWDFIAGGAGRERTLAANEAVFGAVRLRP
RALPGIEEPDTSVEVLGSRWPAPVGIAPVAYHGLAHPDGEPATAAAAGAL
GLPLVVSTFAGRSLEEVARAASAPLWLQLYCFRDHETTLGLARRARDSGY
QALVLTVDTPFTGRRLRDLRNGFAVPAHITPANLTGTAAAGSATPGAHSR
LAFDRRLDWSFVARLGAASGLPVLAKGVLTAPDAEAAVAAGVAGIVVSNH
GGRQLDGAPATLEALPEVVSAVRGRCPVLLDGGVRTGADVLAALALGARA
VLVGRPALYALAVGGASGVRRMLTLLTEDFADTMVLTGHAATGTIGPDTL
APPHHAPPHHGPPTAPRPAPHRDRSHG
Lactate monooxygenase from Mycobacterium
smegmatis
(SEQ ID NO: 15)
MSNWGDYENEIYGQGLVGVAPTLPMSYADWEAHAQQALPPGVLSYVAGGS
GDEHTQRANVEAFKHWGLMPRMLMAATERDLSVELWGKTWAAPMFFAPIG
VIALCAQDGHGDAASAQASARTGVPYITSTLAVSSLEDIRKHAGDTPAYF
QLYYPEDRDLAESFIRRAEEAGYDGLVITLDTWIFGWRPRDLTISNFPFL
RGLCLTNYVTDPVFQKKFKAHSGVEAEGLRDNPRLAADFWHGLFGHSVTW
EDIDWVRSITKMPVILKGIQHPDDARRAVDSGVDGIYCSNHGGRQANGGL
PALDCLPEVVKASGDTPVLFDSGIRTGADVVKALAMGASAVGIGRPYAWG
AALGGSKGIEHVARSLLAEADLIMAVDGYRNLKELTIDALRPTR
Mandelate dehydrogenase from Pseudomonas
putida
(SEQ ID NO: 16)
MSQNLFNVEDYRKLRQKRLPKMVYDYLEGGAEDEYGVKHNRDVFQQWRFK
PKRLVDVSRRSLQAEVLGKRQSMPLLIGPTGLNGALWPKGDLALARAATK
AGIPFVLSTASNMSIEDLARQCDGDLWFQLYVIHREIAQGMVLKALHTGY
TTLVLTTDVAVNGYRERDLHNRFKIPMSYSAKVVLDGCLHPRWSLDFVRH
GMPQLANFVSSQTSSLEMQAALMSRQMDASFNWEALRWLRDLWPHKLLVK
GLLSAEDADRCIAEGADGVILSNHGGRQLDCAISPMEVLAQSVAKTGKPV
LIDSGFRRGSDIVKALALGAEAVLLGRATLYGLAARGETGVDEVLTLLKA
DIDRTLAQIGCPDITSLSPDYLQNEGVTNTAPVDHLIGKGTHA

### Vectors and Host Cells

In another embodiment, provided herein is a vector comprising the engineered lactate oxidoreductase-encoding polynucleotide or a host cell expressing the vector comprising the engineered lactate oxidoreductase-encoding polynucleotide. Engineered lactate oxidoreductase may be prepared by inserting an engineered or mutant polynucleotide into an appropriate expression vector and introducing the vector into an appropriate host cell, such as, for example, Escherichia coli. The transformant is cultured and the engineered lactate oxidoreductase expressed in the transformant may be collected from the cells or culture medium by any known technique.

In embodiments, the engineered lactate oxidoreductase thus obtained may be purified by any of the known purification techniques including, but not limited to, ion exchange column chromatography, affinity chromatography, liquid chromatography, filtration, ultrafiltration, salt precipitation, solvent precipitation, immunoprecipitation, gel electrophoresis, isoelectric electrophoresis and dialysis.

In embodiments, provided herein are isolated or purified polypeptides, proteins and polynucleotides for an engineered lactate oxidoreductase, a vector comprising the polynucleotide encoding the engineered lactate oxidoreductase, a host cell transformed with such a vector, and a method for preparing the engineered lactate oxidoreductase by culturing the transformant, collecting and purifying the engineered lactate oxidoreductase from the culture.

### IV. Devices

In another embodiment, a device for assaying lactate in a sample is provided, where the device includes an engineered lactate oxidoreductase as described herein and optionally an electron mediator.

In one embodiment, biosensor test strips having at least the engineered lactate oxidoreductase as described herein as a reagent are provided. The assay device may have a similar structure as any conventional, commercially available electrochemical (e.g., amperometric) biosensor test strip for monitoring the lactate levels in a sample of non-biological derived or biological derived, such as a blood, a serum, a saliva, a tear, a urine, a sweat or interstitial fluid. One example of such a device has two electrodes (i.e., a working electrode and a reference or counter electrode) positioned on an insulating substrate, a reagent port and a sample receiver. The reagent port contains the engineered lactate oxidoreductase and the electron mediator. In an embodiment, the mediator is potassium ferricyanide, phenazine methosulfate, ferrocene, quinone, osmium, methylene green and derivatives thereof.

In one embodiment, a sample of non-biological derived or biological derived, such as a blood, a serum, a saliva, a tear, a urine, a sweat or interstitial fluid sample, is added to the sample receiver, lactate contained in the sample will react with the engineered lactate oxidoreductase and the electron mediator to generate a current, which is indicative of the amount of lactate in the sample.

In another embodiment, optical detection technologies might be used. Typically, such optical devices are based on color changes that occur in a reagent system comprising an enzyme, an electron mediator, and an indicator. The color changes can be quantified using fluorescence, absorption, transmission, or remission measurements. Examples of optical devices for determining enzyme substrate concentration are known in, for example, U.S. Pat. Nos. 7,008,799; 6,036,919 and 5,334,508.

In another embodiment, provided herein is an enzyme electrode having at least the engineered lactate oxidoreductase immobilized on the electrode. In another embodiment, provided herein is an enzyme sensor for assaying lactate comprising an enzyme electrode as described herein as a working electrode. The concentration of lactate in a sample may be determined by measuring the amount of electrons generated by the enzyme reaction. In embodiments, a sensor system such as carbon (C) electrode, metal electrode, and Pt electrode.

In one embodiment, the engineered lactate oxidoreductase can be immobilized on electrodes. Examples of means for immobilizing molecules such as the engineered lactate oxidoreductase include, but are not limited to, cross-linking, encapsulating into a macromolecular matrix, coating with a dialysis membrane, optical cross-linking polymer, electroconductive polymer, oxidation-reduction polymer, and any combination thereof.

In embodiments, the electrode is a screen printed carbon electrode, a planar gold electrode, or an interdigitated electrode array.

When the measurement is conducted in an amperometric system using a C electrode, gold (Au) electrode or Pt electrode provided with an immobilized enzyme is used as a working electrode, together with a counter electrode (such as a Pt electrode) and a reference electrode (such as an Ag/AgCl electrode). The electrodes can be inserted into a buffer containing a mediator and kept at predetermined temperature.

A predetermined voltage can be applied to the working electrode, and then a sample is added and an increased value in electric current is measured. It is generally also possible to use so-called two-electrode systems with one working electrode and one counter or pseudo-reference electrode.

In another embodiment, lactate may be assayed using an immobilized electron mediator in an amperometric system using a C electrode, Au electrode or Pt electrode. The enzyme, such as an engineered lactate oxidoreductase, can be immobilized on the electrode together with an electron mediator such as potassium ferricyanide, ferrocene, osmium derivative, or phenazine methosulfate in a macromolecular matrix by means of adsorption or covalent bond to prepare a working electrode.

In one embodiment, the working electrode can be inserted into buffer together with a counter electrode (such as a Pt electrode) and a reference electrode (such as an Ag/AgCl electrode), and kept at a predetermined temperature. As indicated above, a predetermined voltage can be applied to the working electrode, and then the sample is added and increased value in electric current is measured.

In one embodiment, the electrode comprises an outer membrane.

Engineered lactate monooxygenases and lactate dehydrogenases provided herein may also be used in devices as described to assay lactate. Alternatively, the engineered enyzmes provided herein may be used in devices as described to assay other molecules. For example, the glycolate oxidases may be used to assay glycolate, long-chain hydroxy acid oxidases may be used to assay long-chain hydroxy acids, and mandelate oxidate may be used to assay mandelate.

### V. Kits

In another embodiment, a kit for assaying lactate in a sample, where the kits include at least an engineered lactate oxidoreductase as described herein and optionally an electron mediator.

Additionally, the kits can include a buffer necessary for the measurement, an appropriate electron mediator and, if necessary, further enzymes such as lactate oxidase, lactate dehydrogenase and flavocytochrome b2, a standard solution of lactate for preparing a calibration curve and an instruction for use. The engineered lactate oxidoreductase may be provided in various forms such as, for example, a freeze-dried reagent or a solution in an appropriate storage solution.

Any or all of the kit reagents can be provided within containers that protect them from the external environment, such as in sealed containers. Positive and/or negative controls can be included in the kits to validate the activity and correct usage of reagents employed in accordance with the inventive concept. Controls can include samples known to be either positive or negative for the presence of a predetermined concentration of lactate.

Engineered lactate monooxygenases and lactate dehydrogenases provided herein may also be used in kits as described for lactate assays. Alternatively, the engineered enyzmes provided herein may be used in kits as described for assays of other molecules. For example, the glycolate oxidases may be used for glycolate assays, long-chain hydroxy acid oxidases may be used for long-chain hydroxy acid assays, and mandelate oxidate may be used for mandelate assays.

### VI. Methods

The engineered lactate oxidoreductases disclosed herein can be used in various methods. For example, they can be used in methods of assaying lactate in a sample from a subject. In embodiments, the sample comprises material selected from the group consisting of blood, serum, saliva, tears (i.e., lacrimal gland secretions), urine, sweat, and interstitial fluid.

The method can include at least a step of contacting the sample with the engineered lactate oxidoreductase and a step of measuring the amount of the lactate oxidized by the engineered lactate oxidoreductase as described above and further below. In embodiments, the method includes continuous measurement of the amount of lactate oxidized by the engineered lactate oxidoreductase. Engineered lactate monooxygenases and lactate dehydrogenases provided herein may also be used in methods as described to assay lactate. These methods may be adapted, mutatis mutandis, for the assay of other substrates modified by the engineered enzymes disclosed herein. For example, the glycolate oxidases may be used in methods of assaying glycolate, long-chain hydroxy acid oxidases may be used in methods of assaying long-chain hydroxy acids, and mandelate oxidate may be used in methods of assaying mandelate.

The disclosed subject matter is further described in the following non-limiting Examples. It should be understood that these Examples, while indicating preferred embodiments of the subject matter, are given by way of illustration only.

### EXAMPLES

### Example 1: Preparation of Recombinant of AvLOx Mutants

pET30c-AvLOx containing WT, A96L, V20CN185C/A96L (A96L CC1), T176C/Q350C/A96L, A68C/M251C/A96L, L67C/D248C/A96L, E72C/Q220C/A96L, V177C/L351C/A96L, or E272C/E303C/A96L was transformed into E. coli BL21(DE3) by heat shock and plated on LB agar medium 50 μg/mL Km and cultivated for 9 hours. After 9 hours, the E. coli was mixed with 3.0 mL LB medium 50 μg/mL Km and precultivated for 8 hours at 37 degrees Celsius. A 30 μl inoculation was made. 3.0 mL ZYP-5052 medium 50 μg/mL Km was added and cultivated for 24 hours at 30 degrees Celsius and 150 r.p.m.

2 mL of culture solution is centrifuged, 10000 g at 4 degrees Celsius for 5 minutes. Wet cells were made in 20 mM Potassium phosphate buffer (P.P.B.) at a pH of 7.0 plus BugBuster. The solution was shaken for 30 minutes at 4 degrees Celsius. The solution was then centrifuged at 15,000 g for 20 minutes at 4 degrees Celsius.

A non-reducing SDS-PAGE analysis and activity assay was performed on the soluble fraction and concertation was determined by Bradford assay.

### Example 2: Analysis of Enzyme Stability of AvLOx Mutants

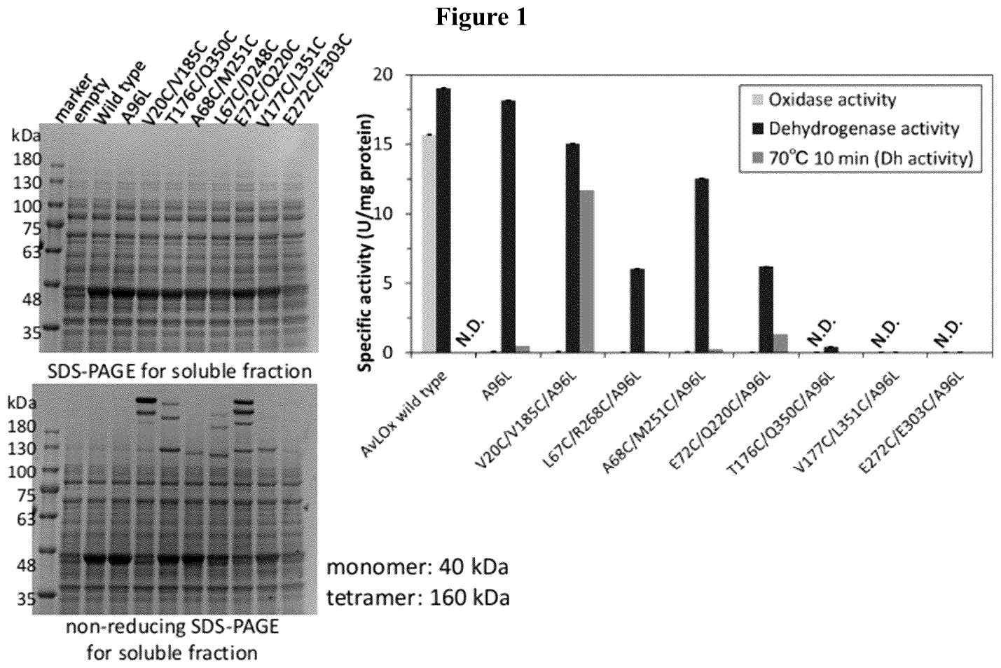

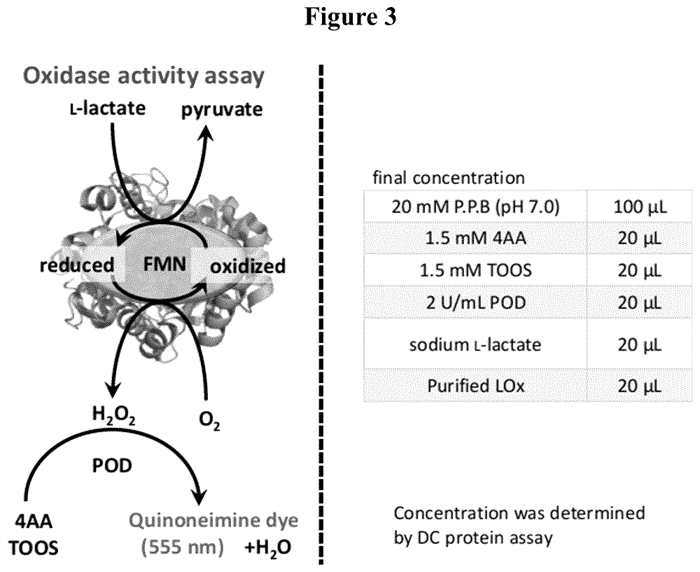

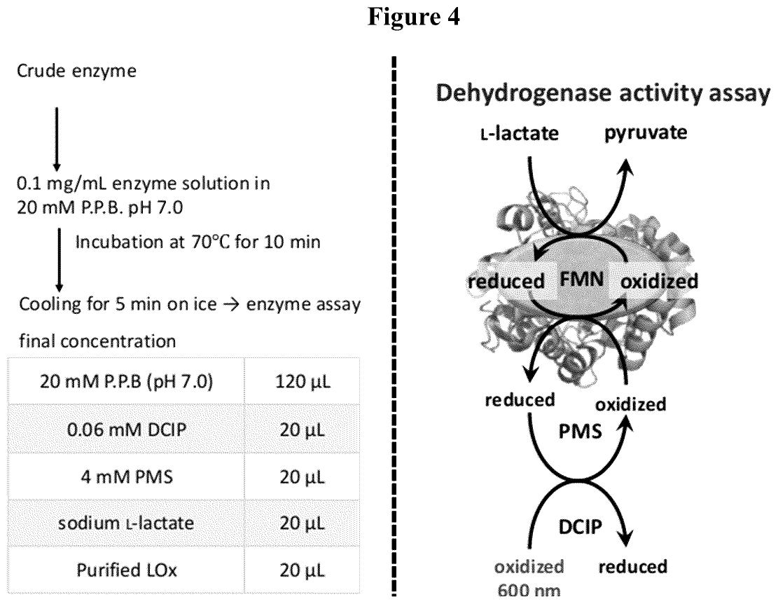

The oxidase and dehydrogenase activity assays are shown in FIGS. 3 and 4. The results are shown in FIG. 1.

The residual activity of the mutants after 70 degree Celsius incubation was also tested. The A96L mutant with the further V20CN185C (CC1) mutations showed residual activity after 70° C. incubation (FIG. 1).

Lastly, SDS-PAGE analysis showed that the CC1 mutant was in multimeric form with disulfide bonds (FIG. 1).

V20CN185C was selected for further experimentation.

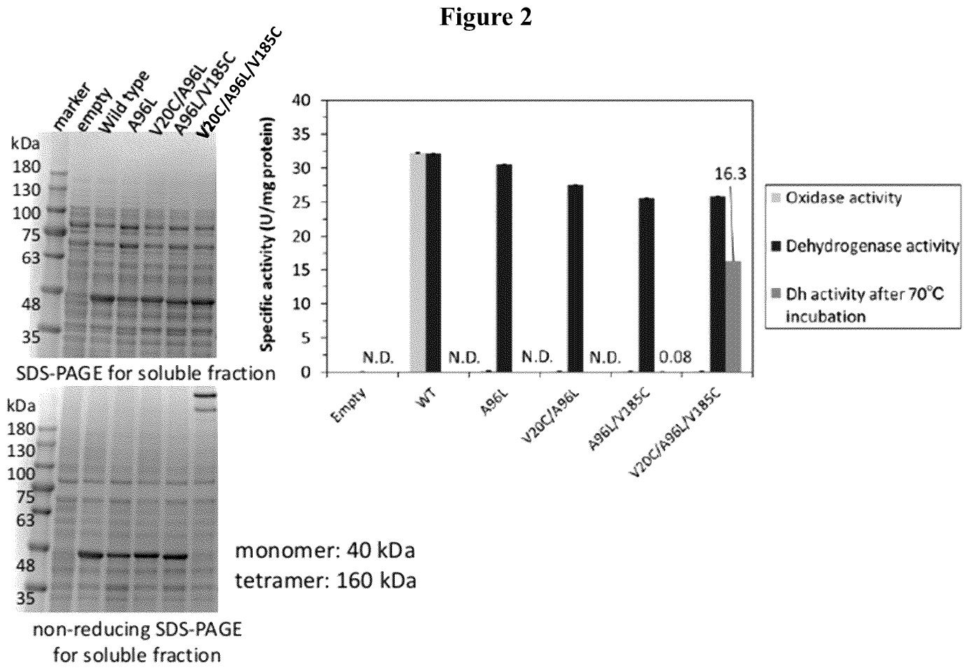

The single cysteine mutation effects were tested. Only the A96L CC1 mutation showed high residual activity after 70° C. incubation and only the CC1 mutant showed a multimeric form with disulfide bond. The V20C/A96L and A96LN185C mutants did not increase LOx stability (FIG. 2). Therefore, the disulfide bridge makes LOx stable. These experiments were carried out using crude cell extract enzyme samples, not purified samples. Accordingly, the SDS-PAGE results are provided to demonstrate that the difference in enzyme activities in the figures on the right side of FIG. 2 are not due to low expression level of enzymes, but due to their own catalytic activities. The SDS-PAGE results show each band at around 50 kDa with almost the same intensities, guaranteeing the same expression level.

### Example 3: Preparation of V20C/V185C/A96L (A96L CC1) AvLOx Mutant

pET30c-AvLOx containing V20CN185C/A96L (A96L CC1) was transformed into E. coli BL21(DE3) by heat shock and plated on LB agar medium 50 μg/mL Km and cultivated for 10 hours. After 10 hours, the bacteria was cultured in 5.0 mL LB medium 50 μg/mL Km and cultivated for 12 hours at 37 degrees Celsius. After 12 hours, the E. coli was mixed with 100 mL×6 ZYP-5052 medium 50 μg/mL Km and cultivated for 36 hours at 30 degrees Celsius at 120 rpm.

The culture solution was centrifuged, 5000 g at 4 degrees Celsius for 10 minutes. The solution was harvested and washed twice in 0.85% NaCl. Wet cells were made in 20 mM Potassium phosphate buffer (P.P.B.) at a pH of 7.0. The solution was run through a French press and then centrifuged at 10,000 g for 20 minutes at 4 degrees Celsius and then 100,000 g for 60 minutes at 4 degrees Celsius.

The soluble fraction was then dialyzed against 20 mM P.P.B. The dialysate purified using AKTA FPLC system, Anion Exchange Chromatography (ResourceQ) with A buffer: 20 mM P.P.B. (pH 7.0) and B buffer: 0.5 M KCl, 20 mM P.P.B. (pH 7.0). The dialysate purified with a linear gradient of 0-0.5 M KCl. The 455 nm peak fraction from oxidized FMN was run through Amicon Ultra-15 (50K) filter and concentrated and desalted. The purified fraction was collected.

### Example 4: Analysis of A96L CC1 Mutant

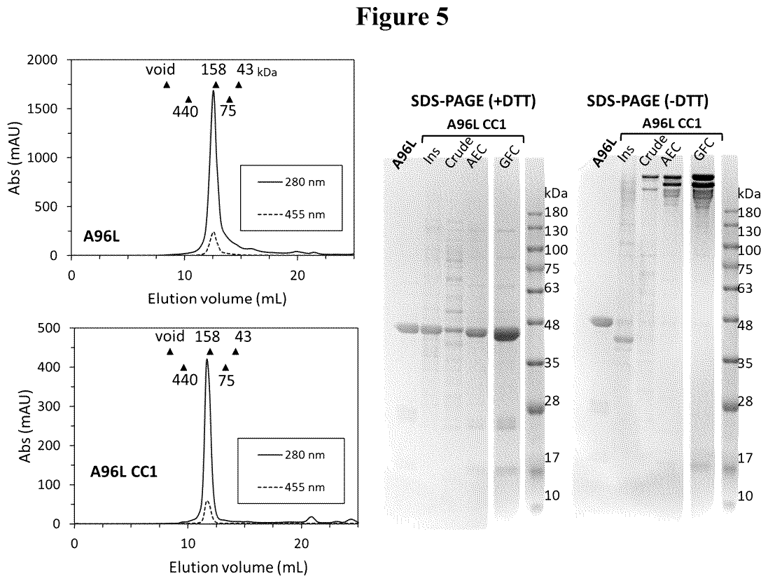

The A96L CC1 mutant was analyzed. Gel filtration chromatography was performed and results are shown in FIG. 5. The results showed that the A96L CC1 mutant formed tetramers in solution.

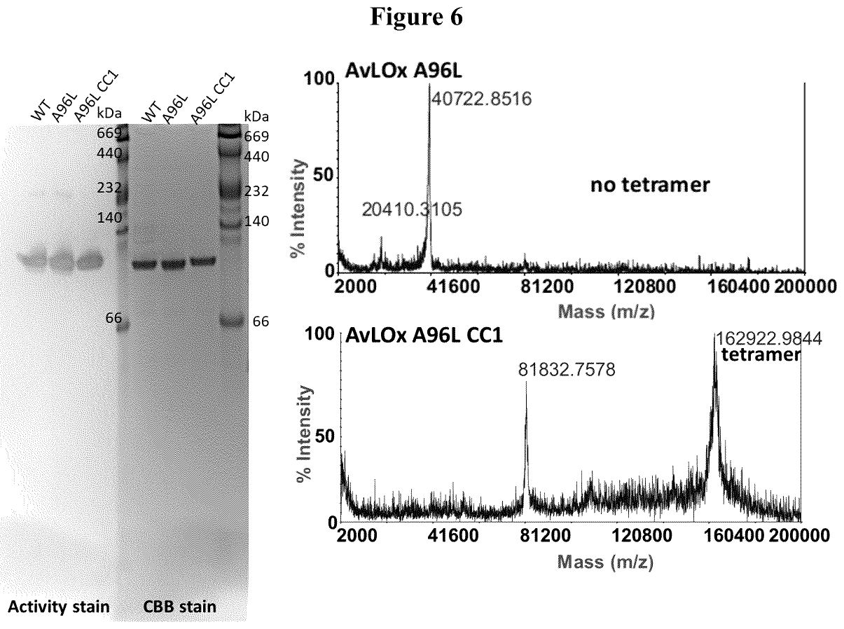

Furthermore, a native-PAGE/TOF-MS analysis was performed, and results are shown in FIG. 6. The A96L CC1 mutant showed almost the same molecular weight as the A96L mutant in Native-PAGE. However, the A96L mutant showed no tetramer formation while the A96L CC1 mutant showed tetramer formation.

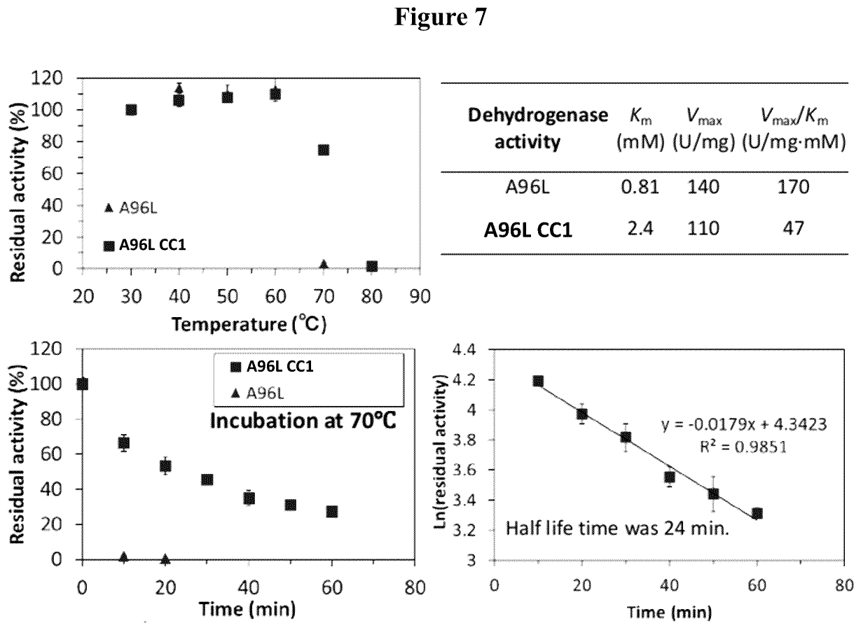

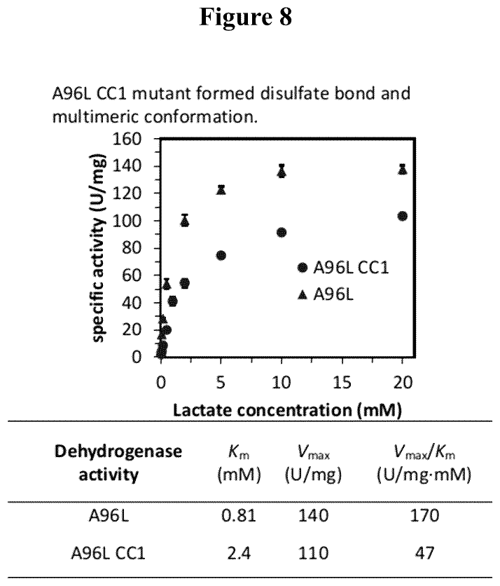

A96L mutant and A96L CC1 mutant activity and stability was tested. Results are shown in FIG. 7. A96L CC1 showed high thermal stability compared to A96L. Further kinetic parameter and substrate specificity analysis were performed, and the results are shown in FIG. 8.

A96L CC1 mutant (V20CN185C/A96L) formed disulfide bonds and a multimeric conformation.

### Example 5: Fusion Enzyme Preparation and Analysis

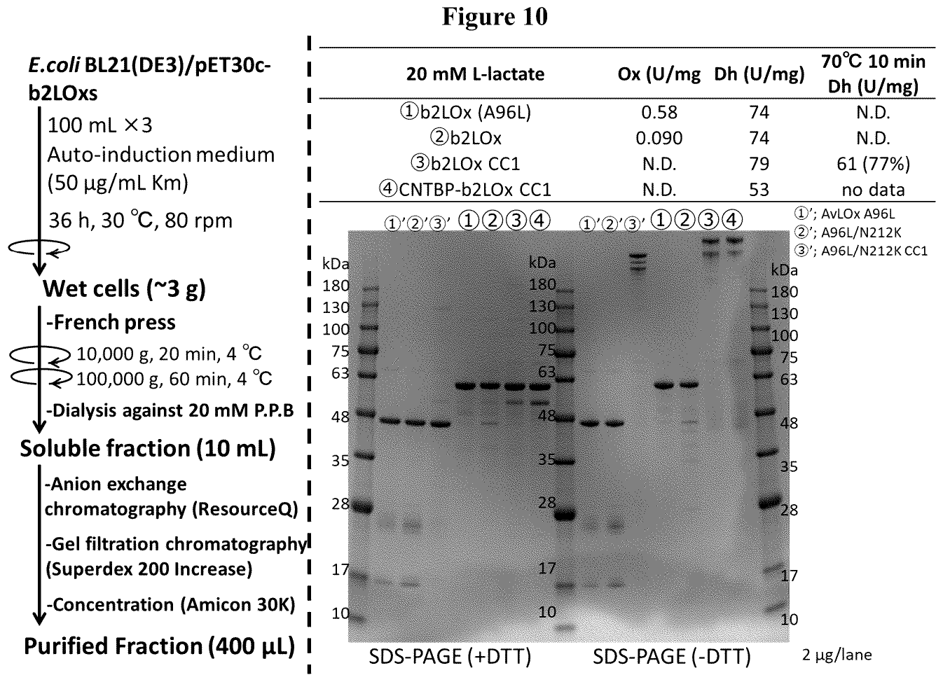

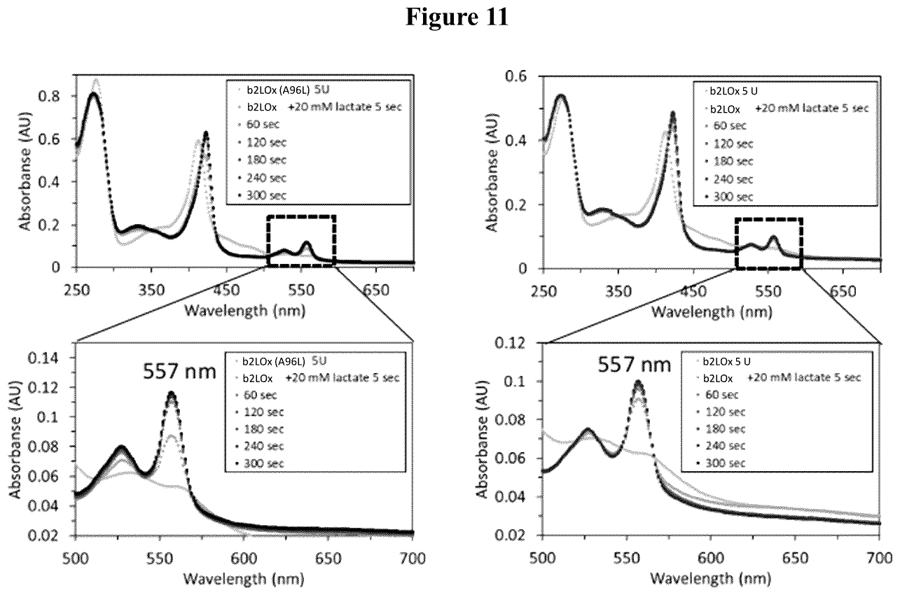

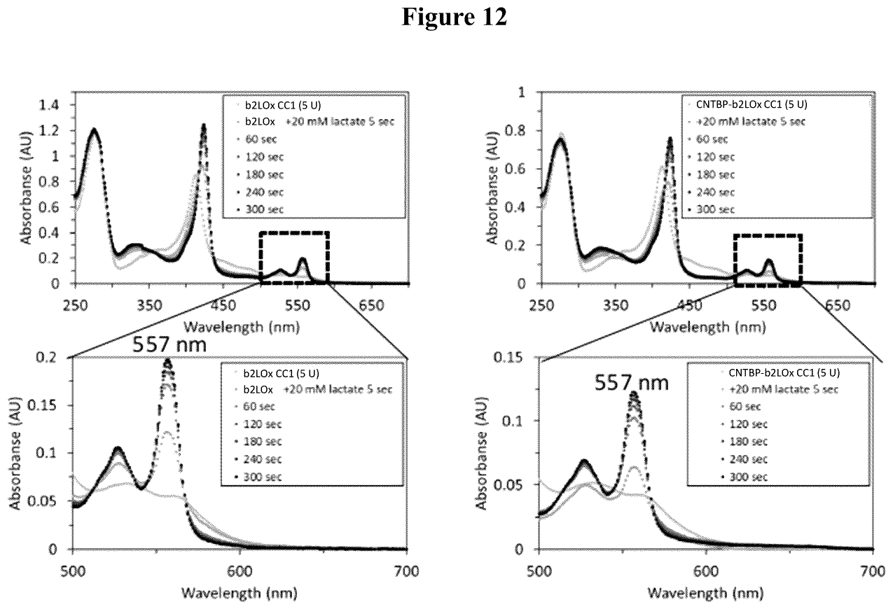

Fusion enzyme with AvLOx A96L/N212K and Pichia pastoris flavocytochrome b2 heme domain was prepared (FIG. 10). SDS-PAGE results are shown in FIG. 10. These results were obtained under two different conditions; under reduced conditions (left) and under non-reduced conditions (right). Under the non-reducing conditions, lanes 3 and 4 with CC mutants, which are expected to contain a disulfide bond, migrated differently compared with the same mutants under reducing conditions. The results show that the disulfide bonds were formed in these mutants. Absorption spectra of the exemplary electrodes were measured (FIGS. 11 and 12).

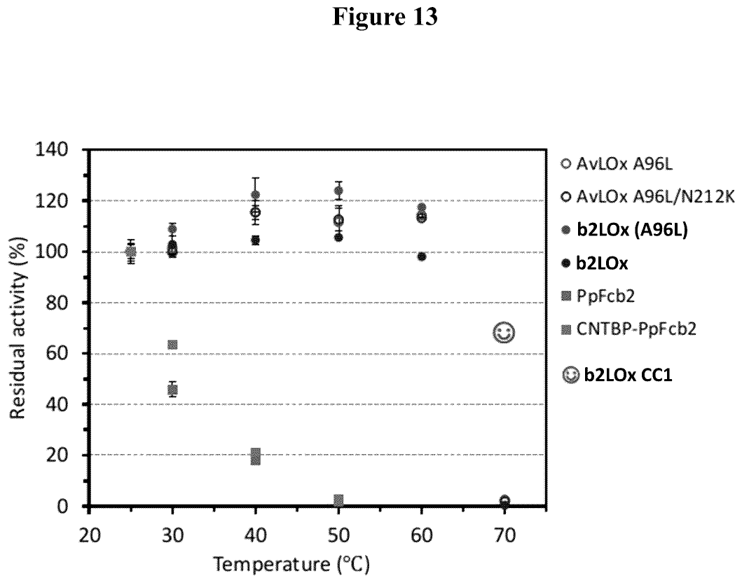

Temperature stability of the FcbLOx A96L/N212K CC1 mutant was tested. The FcbLOx A96L/N212K CC1 mutant showed more than 70% of initial activity after 10 min incubation at 70° C. (FIG. 13).

### Example 6: Analysis of PES-Modified Enzyme Electrode

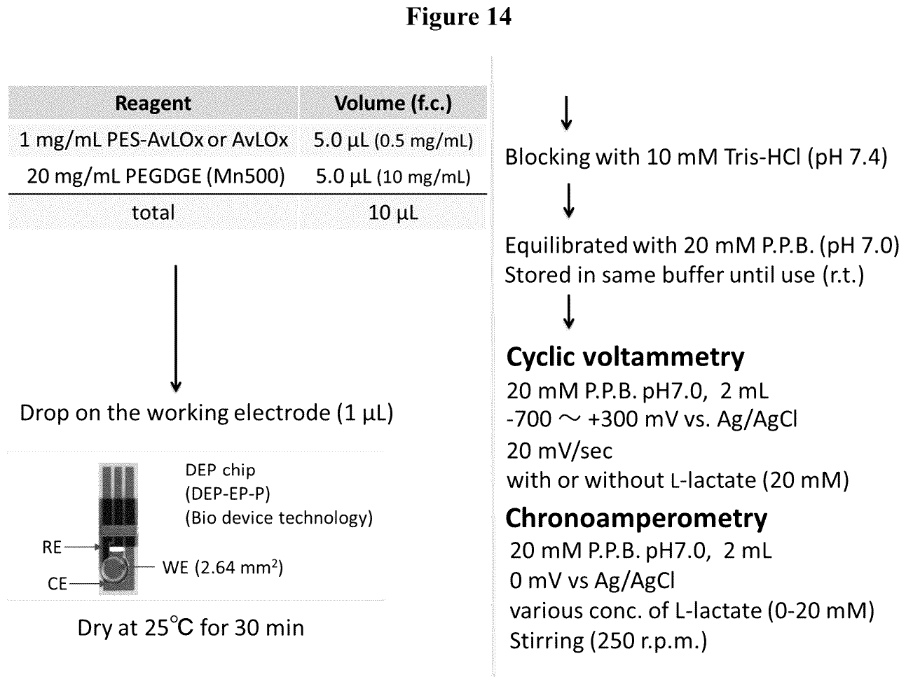

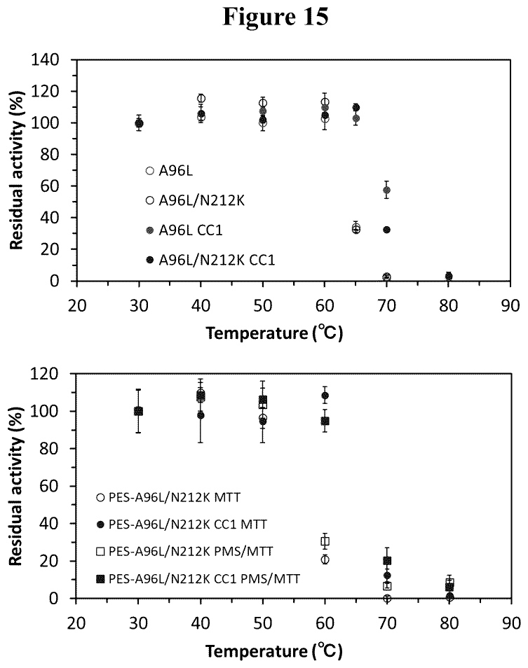

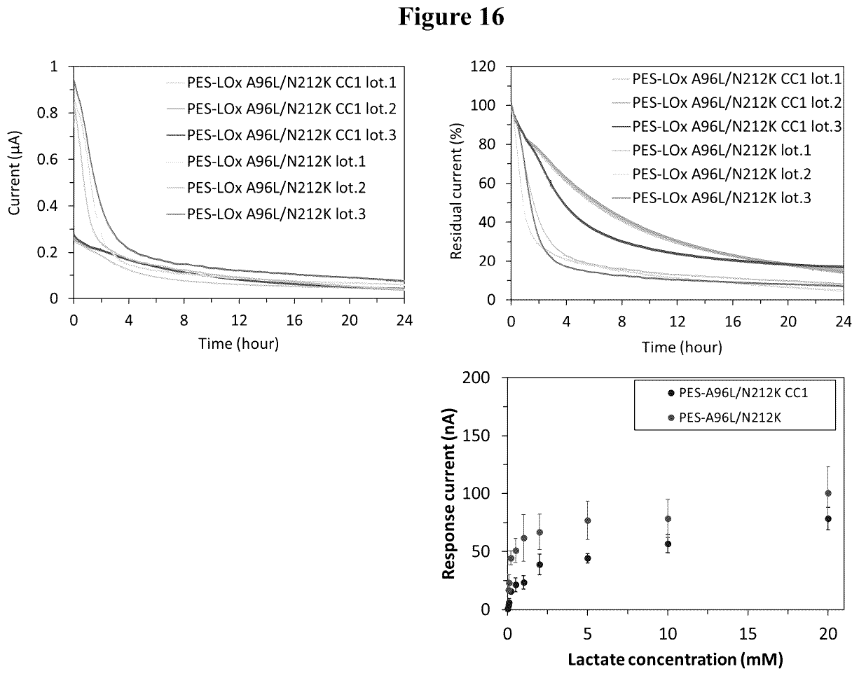

Exemplary phenazine ethosulfate- (PES-) modified wild-type, A96L, N212K, and A96L/N212K electrodes were prepared according to FIG. 14. SDS-PAGE results are shown in FIG. 14. Absorption spectra of the exemplary electrodes were measured (FIGS. 15 and 16).

Results from stability tests of PES-modified CC1 mutants are shown in FIG. 15. CC1 mutants maintained more than 30% residual activity. The other mutants and wild type showed inactivation at 70° C. The A96L/N212K CC1 mutant showed whole residual activity after 60° C. incubation. The A96L/N212K CC1 mutation increased the thermal stability of the enzyme. PES modification may decrease the thermal stability of enzymes due to changing the surface properties of the enzyme.

Results from continuous lactate monitoring using PES-modified CC1 mutant are shown in FIG. 16. Continuous lactate monitoring was tested using 2 mM L-lactate and 200 mV vs. Ag/AgCl. PES-LOx A96L/N212K response current decreased drastically after one hour. PES-LOx A96L/N212K CC1 response current was maintained at more than 50% after 6 hours. Therefore, the CC1 mutant was suitable for continuous lactate monitoring.

### Example 7: Preparation and Analysis of b2LOX and b2LOxS

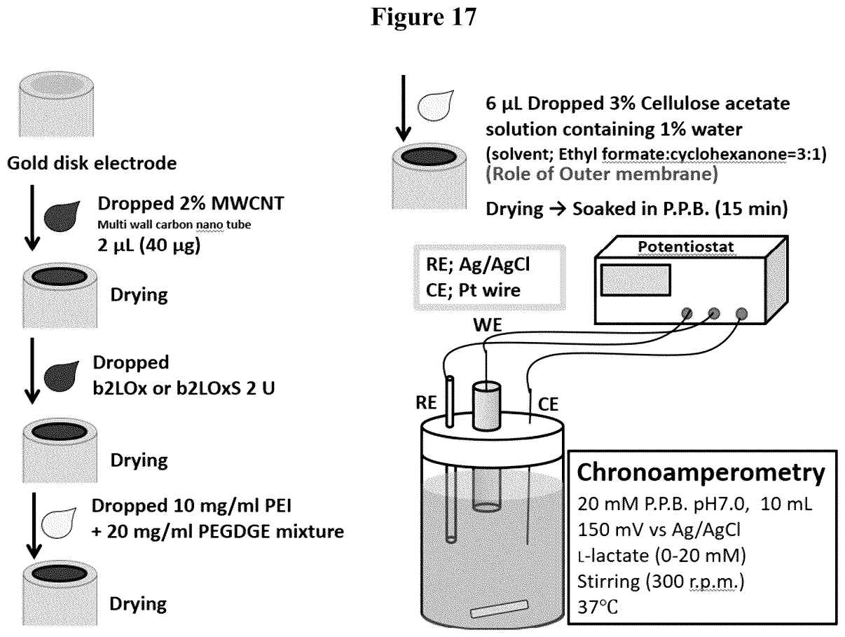

b2LOX and b2LOxS electrodes were prepared according to FIG. 17. b2LOX is a fusion enzyme with the cytochrome domain of flavocytochrome b2. b2LOxS is a fusion enzyme with the cytochrome domain of flavocytochrome b2 fusion enzyme that comprises an Ala95Ser mutation.

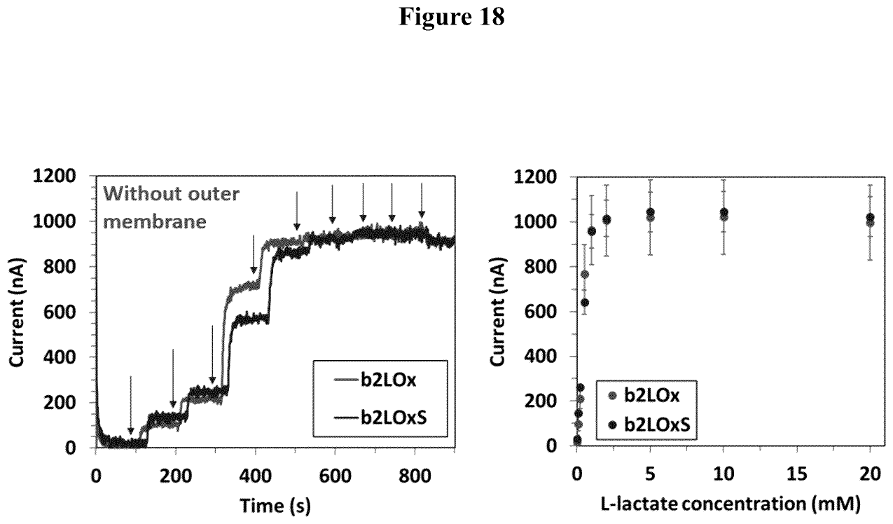

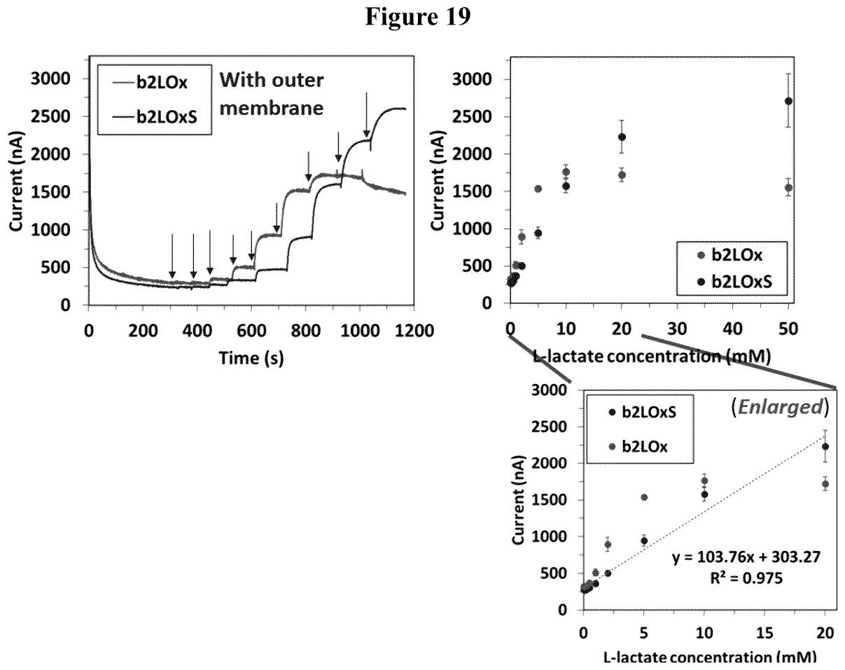

Results of chronoamperometry are shown in FIG. 18. b2LOxS improved the dynamic range of measurement, however, this improvement is not enough to cover physiological range (5-20 mM) lactate concentration. Therefore, next, we used an outer membrane to increase the substrate diffusion layer. The results are shown in FIG. 19.

Use of an outer membrane improved linear range, but the b2LOx electrode still showed signal saturation at lower lactate concentration (less than 10 mM) together with substrate inhibition at high lactate concentration. The electrode with b2LOxS was able to measure lactate concentration into 50 mM, covering physiological lactate concentration without substrate inhibition. b2LOxS achieved broad linear range (0.1-20 mM lactate, R²=0.975). The b2LOxS and outer membrane combination showed improved linear range.

The results indicated that the use of a fusion enzyme such as b2LOX makes it possible to use the enzyme for a direct electron transfer (DET) type lactate sensor. Additionally, use of the A95S mutant, i.e., b2LOxS, which has a larger Km with an outer membrane, showed that the sensor could monitor lactate in the physiological range.

### Example 8: Effect of A95 Mutations in Combination with A96L/N212K Mutations

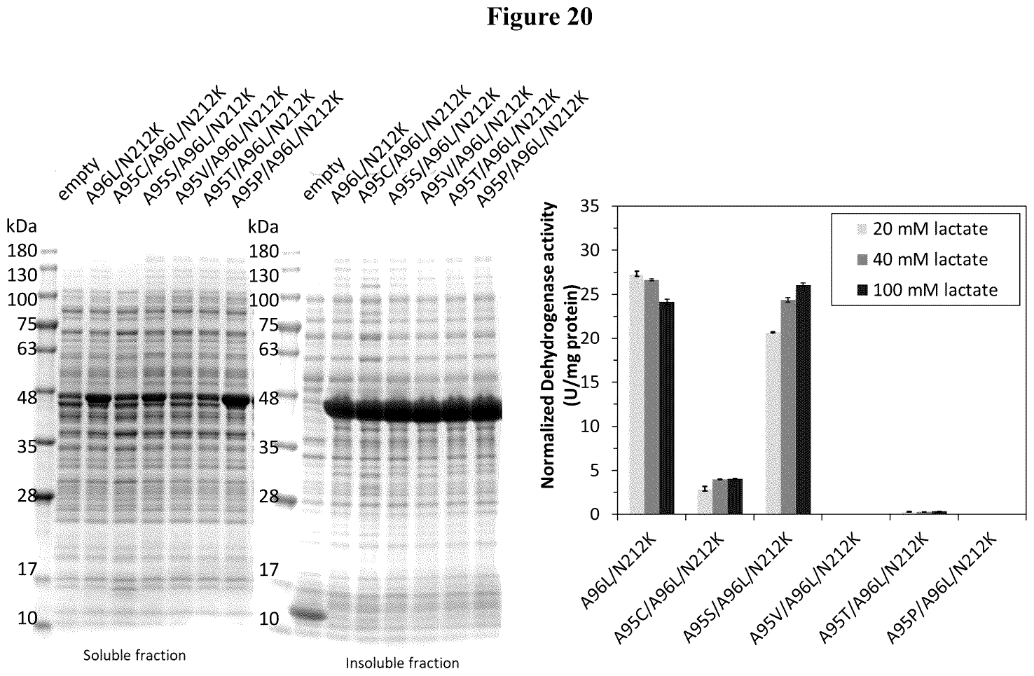

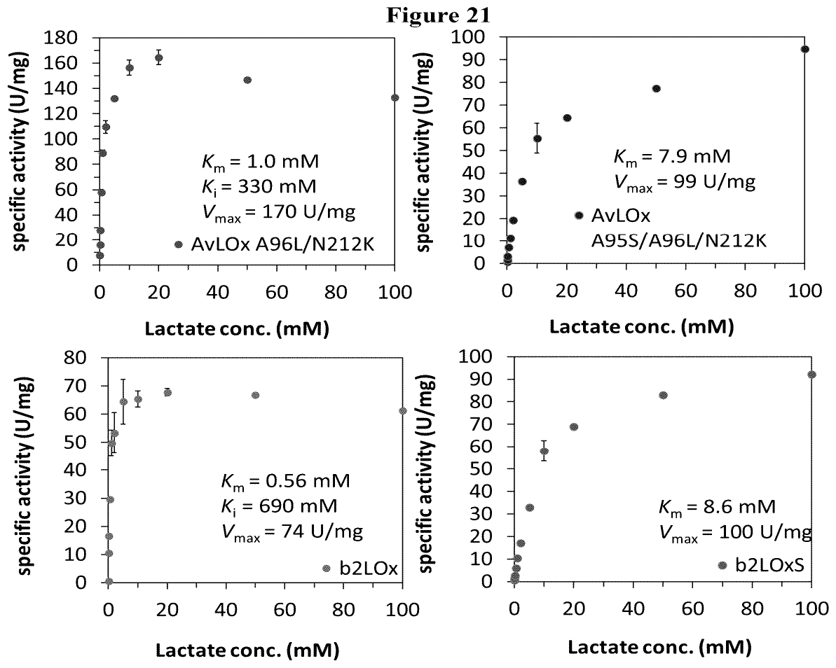

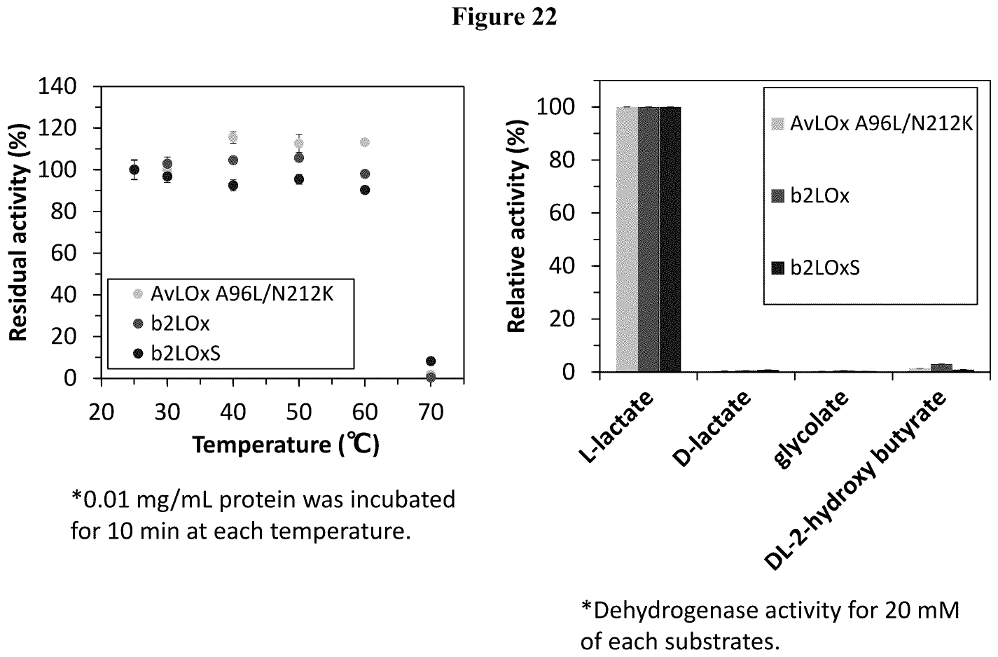

Lactate oxidoreductases with the A96L/N212K mutation, and various A95 mutations, were examined for their ability to suppress substrate inhibition. The A95 mutations included A95C, A95S, A95V, A95T, and A95P. The results are shown in FIG. 20. The A95S/A96L/N212K mutant was the only one to exhibit a similarly high level of dehydrogenase activity as the A96L/N212K mutant. Notably, as lactase concentration increased, the activity for the A96L/N212K mutant decreased. However, as lactase concentration increased, the activity for the A95S/A96L/N212K mutant increased, indicating that this mutant suppressed substrate inhibition. This is shown in more detail in FIG. 21; in the A96L/N212K and the b2LOx fusion enzyme, specific activity peaks at 20 mM lactate, and decreases as lactate concentration increases. In their counterparts with an A95S mutation (A95S/A96L/N212K and b2LOxS, respectively), specific activity continues to increase as lactate concentration increases, to at least a concentration of 100 mM. FIG. 22 shows that the A95S mutation did not affect stability or substrate specificity. As in FIG. 2, the SDS-PAGE results in FIG. 20 make clear that the differences in enzyme activity are not due to differences in expression level, but to their catalytic activities.

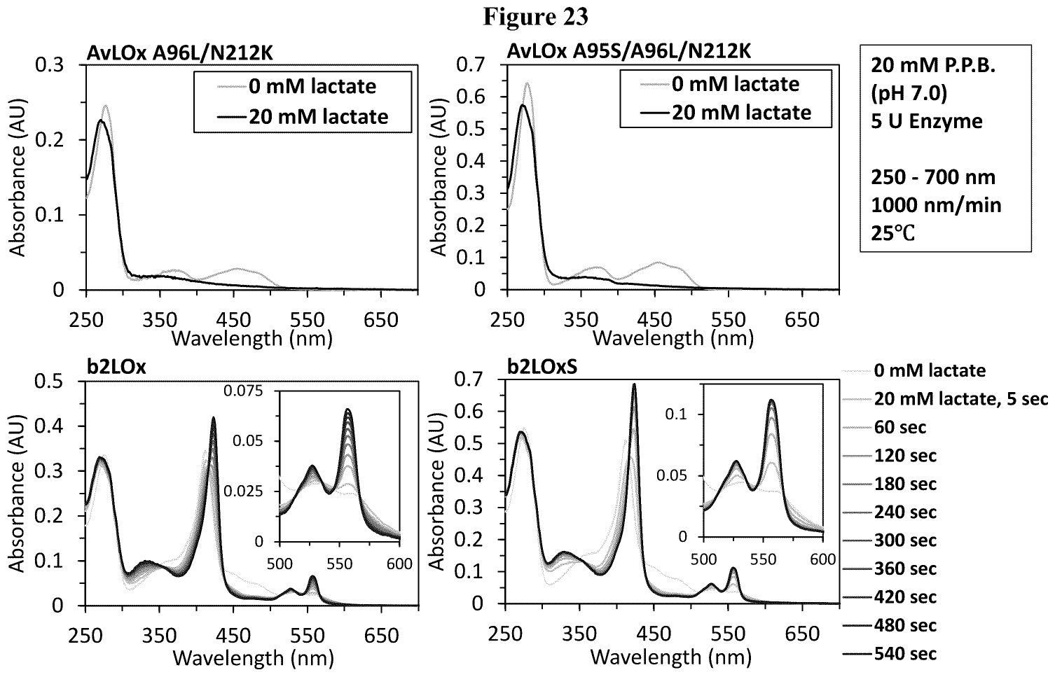

FIG. 23 shows shows the intra-molecular electron transfer of mutants, which was observed by typical spectrum of reduced heme b at increase in 552 nm, in the two bottom spectra (fusion proteins), but not in the upper spectrum (mutant enzymes not harboring fused heme (b2)).

In this investigation, spectroscopic observation of enzymes (either with fusion cytochrome b2 or without), harboring mutation, Ala95Ser, were investigated. The control experiments showing without Ala95Ser (AvLOx A96L/N212K and b2LOx), revealed the fusion of b2 only showed the increase of the 562 nm peak after the addition of lactate, which indicates that the addition of lactate reduces its cofactor FMN, and then transfers electrons from FMN to the oxidized form of fused cytochrome b2, resulting in the reduced form of cytochrome b2 showing its typical absorbance peak at 562 nm, demonstrating that intramolecular electron transfer from FMN to b2 occurred. A similar observation was confirmed in b2LOxS (b2LOx with an Ala95Ser mutation), where an absorbance peak at 562 nm only developed after the addition of lactate, indicating this mutation does not negatively impact the fusion protein's ability to facilitate a DET-type reaction.

### Example 9: Lactate Sensor Employing b2LOxS in Combination with Glucose Sensor

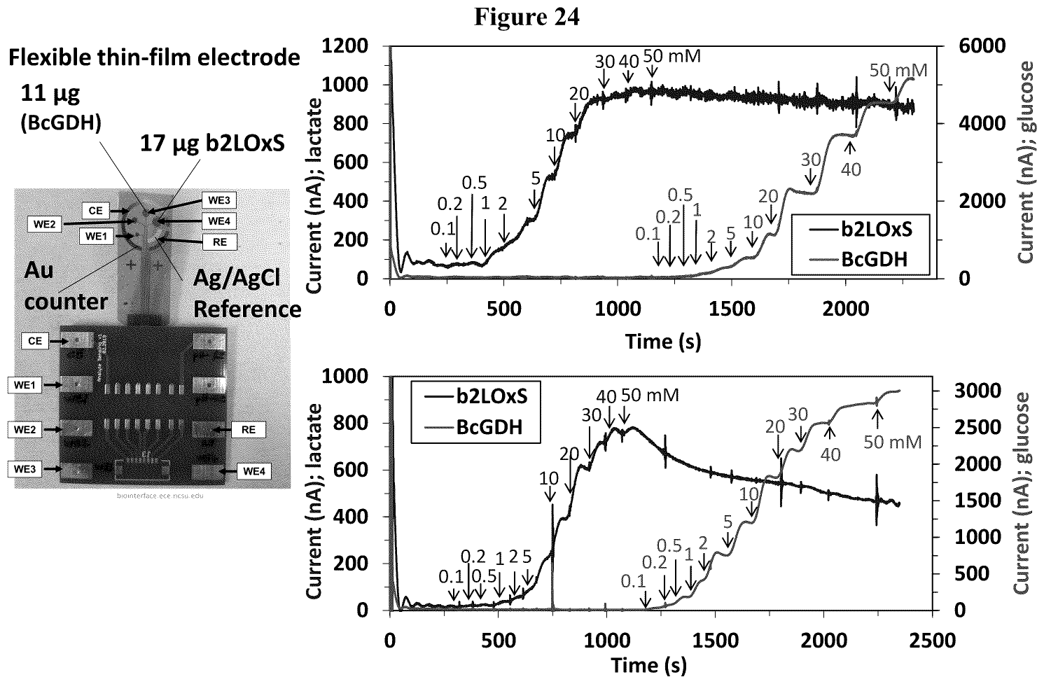

A sensor employing b2LOxS (for monitoring lactate concentration) and glucose dehydrogenase from Burkholderia cepacia (BcGDH) (for monitoring glucose concentration) was tested, to determine if lactate and glucose concentrations could be monitored simultaneously. The sensor contained a flexible thin-film electrode with 17 μg b2LOxS and 11 μg BcGDH, using an Au counter and an Ag/AgCl reference (FIG. 24). The testing was performed on 10 mL of 20 mM PPB solution at pH 7.0 (150 mV vs. Ag/AgCl, 37° C.) and 10 mL of artificial sweat (0.5 w/v % NaCl (f.c. 86.5 mM); 0.1 w/v % KCl (f.c. 13.4 mM); 0.1 w/v % Urea (f.c. 16.7 mM); 2 mM D-lactate; pH was adjusted to 5.4 by 1 mM NH₄OH), with differing concentrations of L-lactate (0-50 mM) and D-glucose (0-10 mM). See Liu et al., Appl. Phys. Lett. (2015) and Kondoh et al., Eur. J. Appl. Physiol. (1992)).

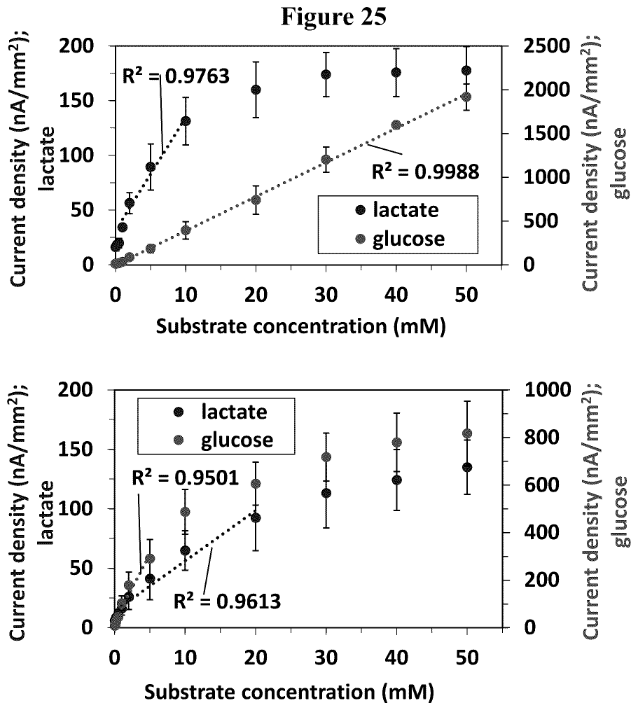

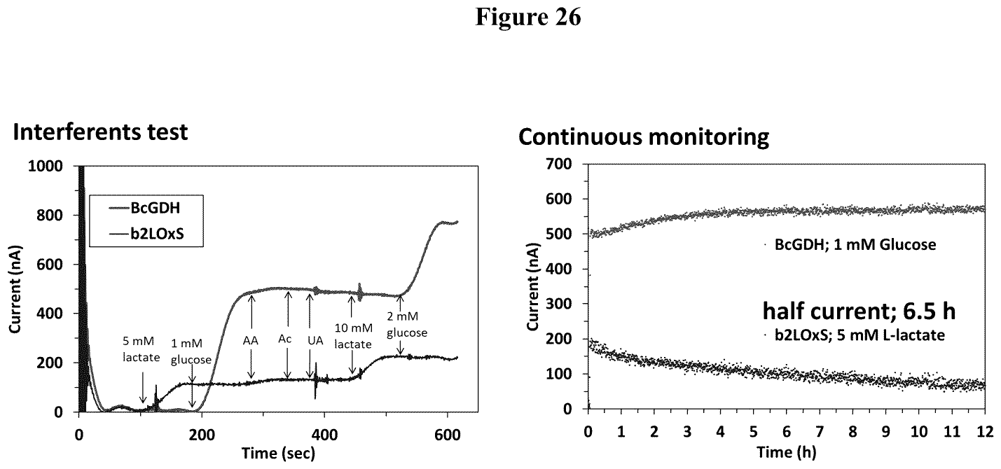

The resulting currents measured by the sensor are shown in FIG. 24. FIG. 25 demonstrates a strong correlation between measured currents and concentrations of L-lactate and D-glucose, in both the PPB solution (top) and the artificial sweat (bottom). FIG. 26, on the left side, demonstrates that the DET-type lactate monitoring based on b2LOxS enzyme was not affected by the presence/addition of ascorbic acid (AA), acetoaminophen (AC), and uric acid (UA) in the artificial sweat. The right side of FIG. 26 shows the stability of DET-type lactate sensor employing b2LOxS enzyme. The half-life at 37° C. was 6.5 hours.

### Example 10: Intra-Subunit Disulfide Bonds and Combinations with Inter-Subunit Disulfide Bonds

Possible intra-subunit disulfide formation to improve enzyme stability was investigated, by picking potential pairs of residues to be substituted with Cys residues. The tested enzyme mutants were A96L/N212K (lacking a CC mutation), A96L/N212K CC1 (inter-subunit CC), and A96L/N212K in combination with each of G46C/Y271C, T50C/A267C, F129C/D164C, K149C/K219C, S175C/Q269C, Q244C/P274C, and A249C/I283C (intra-subunit CCs).

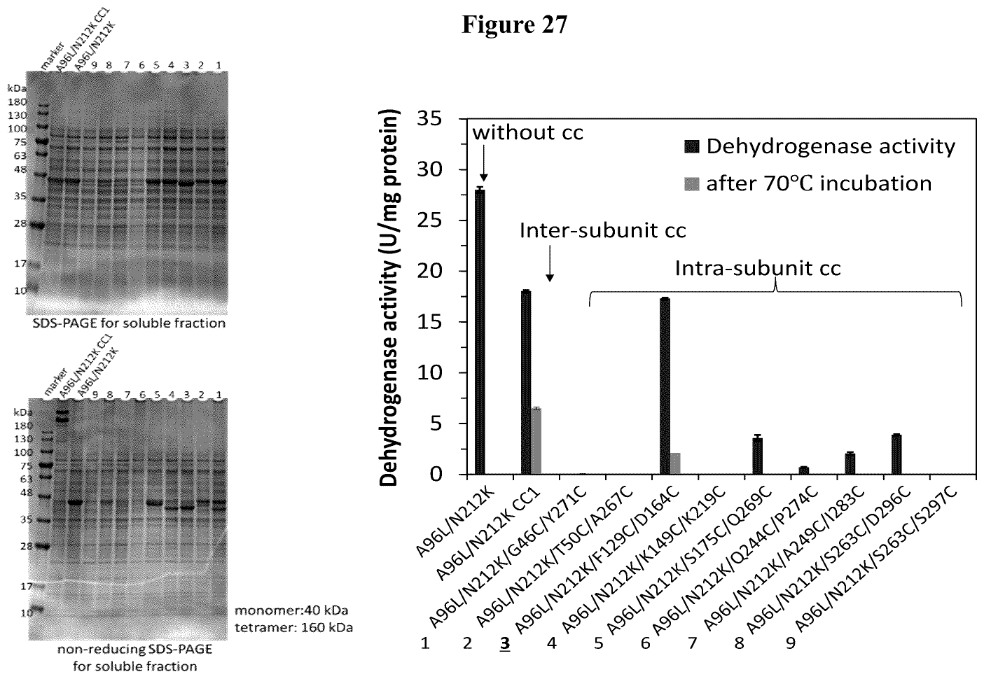

Among the predicted and mutated pairs, only Phe129Cys/Asp164Cys (F129C/D164C) increased thermal stability, as the F129C/D164C mutant alone kept its activity after 70° C. incubation for 10 min. (FIG. 27). Hereinafter, F129C/D164C is designated as “CC2”. As in FIG. 2, the SDS-PAGE results in FIG. 27 make clear that the differences in enzyme activity are not due to differences in expression level, but to their catalytic activities.

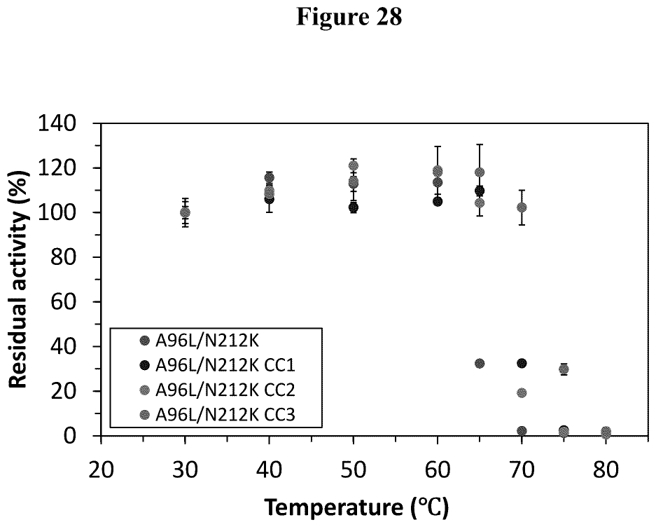

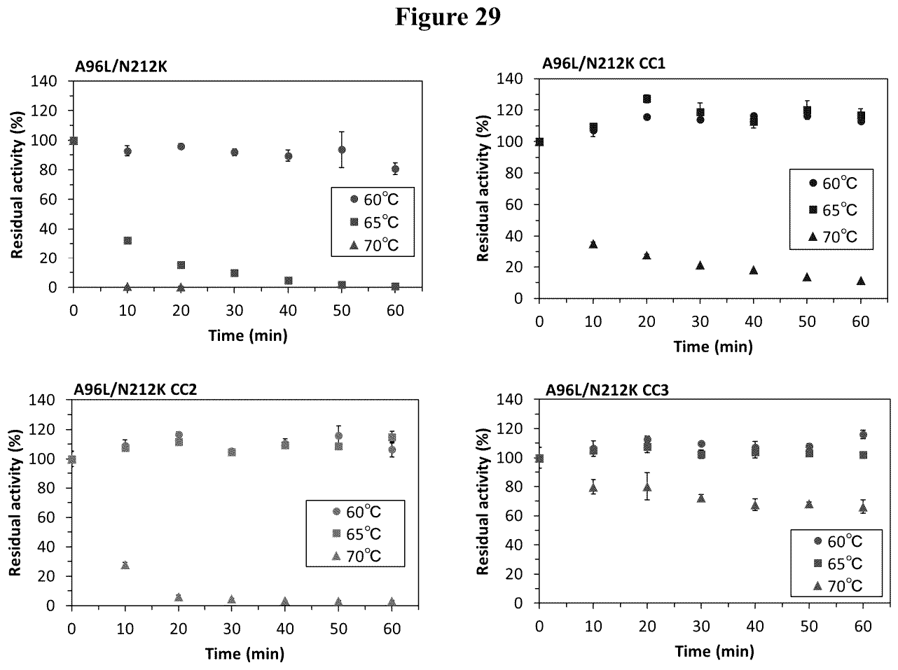

The combination of CC2 (F129C/D164C) with CC1 (V20CN185C), making the mutant V20CN185C/F129C/D164C, hereinafter designated as “CC3,” shows much higher thermal stability than CC1 and CC2. These experiments were carried out with the combination of mutant A96L/N212K, which shows negligible oxidase activity but maintains dye-mediated dehydrogenase activity. The parental enzyme, A96L/N212K, almost entirely lost its activity after incubation at 70° C. for 10 min. A96L/N212K CC3 keeps 80% of activity even after incubation at 70° C. for 10 min, whereas A96L/N212K CC1 keeps 30% and A96L/N212K CC2 keeps 10% of their activity after incubation at 70° C. for 10 min. These facts support the combination of CC2 (F129C/D164C) and CC1 (V20CN185C) mutations as yielding the most thermally stable lactate oxidoreductase. FIG. 28 shows enzymatic activity at different temperatures for the tested mutants. FIG. 29 shows the thermal inactivation of these mutants.

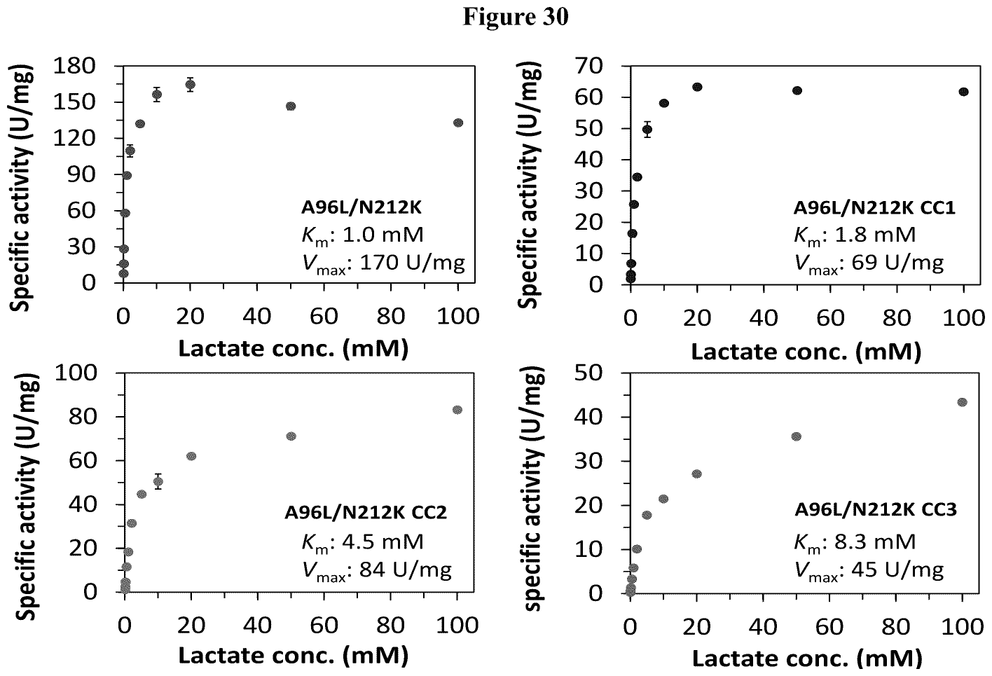

FIG. 30 shows that CC2 and CC3 harbor enough comparably high enzyme activity. In addition, unexpectedly, CC2 mutations, including CC3, result in the elimination of substrate inhibition, as their activities increase at lactate concentrations higher than 20 mM, whereas A96L/N212K and wild type enzyme have their enzyme activities start to decrease at this concentration, due to substrate inhibition.

The combination of CC2 (Phe129Cys/Asp164Cys; F129C/D164C) with CC1 (V20CN185C), that is the mutant V20CN185C/F129C/D164C, which is now designated as CC3, shows much higher thermal stability than CC1 and CC2. These experiments were carried out with the combination of mutant A96L/N212K, which shows negligible oxidase activity but maintains dye-mediated dehydrogenase activity. The parental enzyme, A96L/N212K almost entirely lost its activity after the incubation at 70° C. for 10 minutes. A96L/N212K CC3 keeps 100% of activity even after the incubation at 70° C. for 10 minutes, and more than 66% even after 60 min (FIG. 30). A96L/N212K CC1 keeps 38% and A96L/N212K CC2 keeps 28% of their activity after incubation at 70° C. for 10 minutes. These facts support the combination of CC2 (Phe129Cys/Asp164Cys) and CC1 (V20CN185C) mutations as yielding the lactate oxidoreductase with the highest stability.

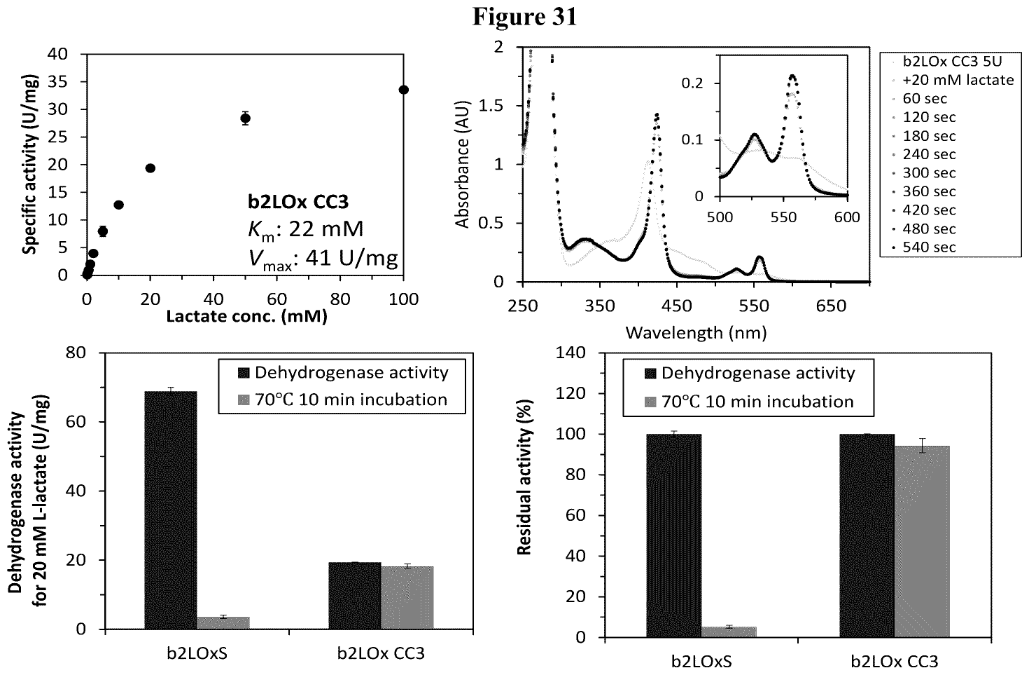

FIG. 31 shows that the CC3 mutation also increases the thermal stability of the fusion lactate oxidase (b2LOx). While b2LOxS has higher L-lactate dehydrogenase activity, initially, b2LOx CC3 has much higher dehydrogenase activity after incubation at 70° C. for 10 minutes. It can be seen that following the incubation, b2LOxS only retains about 5% residual activity, while b2LOx CC3 retains about 95% residual activity. As seen in the top portion of FIG. 31, the b2LOx CC3 mutant also exhibits an increased Kₘ value, and no substrate inhibition. The absorption spectra in the top right of FIG. 31 also demonstrates that the CC3 mutation does not have a negative impact on intramolecular electron transfer, for the same reasons as discussed in connection with FIG. 2.

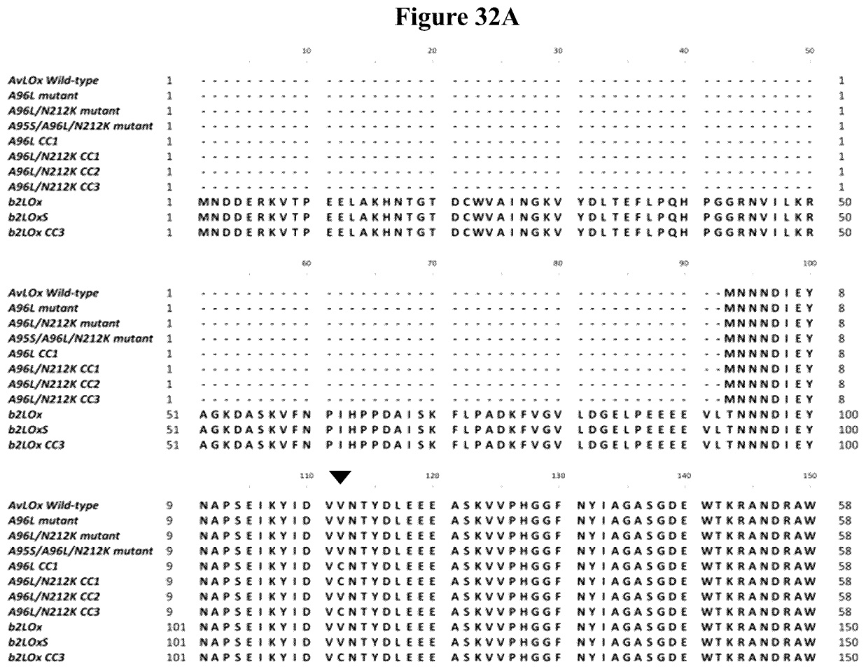

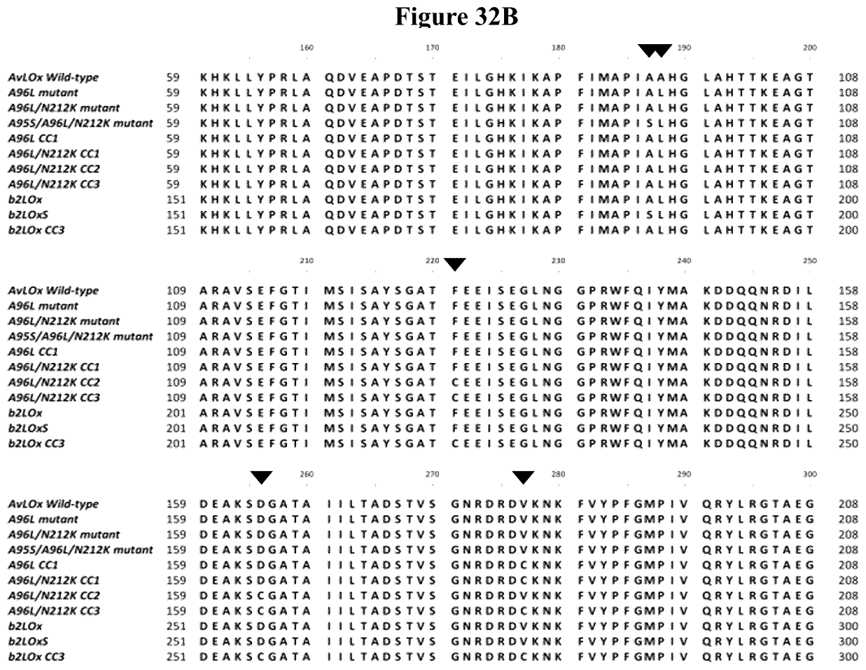

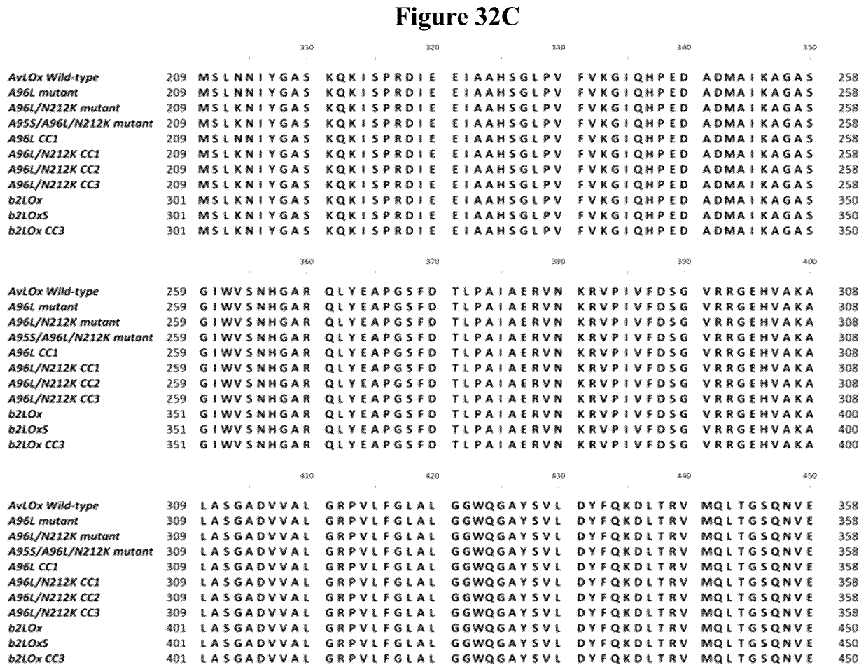

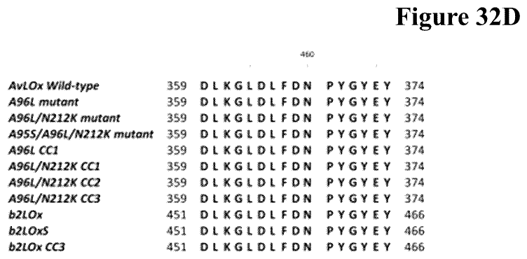

FIGS. 32A-D illustrate the amino acid sequence alignment of some of the main AvLOx mutations and fusion proteins discussed hereinabove, including AvLOx wild type (SEQ ID NO: 1), the A96L mutant (SEQ ID NO: 17), the A96L/N212K mutant (SEQ ID NO: 18), the A95S/A96L/N212K mutant (SEQ ID NO: 19), the A96L CC1 mutant (SEQ ID NO: 20), the A96L/N212K CC1 mutant (SEQ ID NO: 21), the A96L/N212K CC2 mutant (SEQ ID NO: 22), the A96L/N212K CC3 mutant (SEQ ID NO: 23), the b2LOx fusion protein (SEQ ID NO: 5), the b2LOxS mutant (SEQ ID NO: 24), and the b2LOx CC3 mutant (SEQ ID NO: 25). Arrows indicate candidates for cysteine mutation sites. Graphic view was prepared by BioEdit software ver. 7.2.6.

A96L mutant
(SEQ ID NO: 17)
MNNNDIEYNAPSEIKYIDVVNTYDLEEEASKVVPHGGFNYIAGASGDEWT
KRANDRAWKHKLLYPRLAQDVEAPDTSTEILGHKIKAPFIMAPIALHGLA
HTTKEAGTARAVSEFGTIMSISAYSGATFEEISEGLNGGPRWFQIYMAKD
DQQNRDILDEAKSDGATAIILTADSTVSGNRDRDVKNKFVYPFGMPIVQR
YLRGTAEGMSLNNIYGASKQKISPRDIEEIAAHSGLPVFVKGIQHPEDAD
MAIKAGASGIWVSNHGARQLYEAPGSFDTLPAIAERVNKRVPIVFDSGVR
RGEHVAKALASGADVVALGRPVLFGLALGGWQGAYSVLDYFQKDLTRVMQ
LTGSQNVEDLKGLDLFDNPYGYEY
A96L/N212K mutant
(SEQ ID NO: 18)
MNNNDIEYNAPSEIKYIDVVNTYDLEEEASKVVPHGGFNYIAGASGDEWT
KRANDRAWKHKLLYPRLAQDVEAPDTSTEILGHKIKAPFIMAPIALHGLA
HTTKEAGTARAVSEFGTIMSISAYSGATFEEISEGLNGGPRWFQIYMAKD
DQQNRDILDEAKSDGATAIILTADSTVSGNRDRDVKNKFVYPFGMPIVQR
YLRGTAEGMSLKNIYGASKQKISPRDIEEIAAHSGLPVFVKGIQHPEDAD
MAIKAGASGIWVSNHGARQLYEAPGSFDTLPAIAERVNKRVPIVFDSGVR
RGEHVAKALASGADVVALGRPVLFGLALGGWQGAYSVLDYFQKDLTRVMQ
LTGSQNVEDLKGLDLFDNPYGYEY
A95S/A96L/N212K mutant
(SEQ ID NO: 19)
MNNNDIEYNAPSEIKYIDVVNTYDLEEEASKVVPHGGFNYIAGASGDEWT
KRANDRAWKHKLLYPRLAQDVEAPDTSTEILGHKIKAPFIMAPISLHGLA
HTTKEAGTARAVSEFGTIMSISAYSGATFEEISEGLNGGPRWFQIYMAKD
DQQNRDILDEAKSDGATAIILTADSTVSGNRDRDVKNKFVYPFGMPIVQR
YLRGTAEGMSLKNIYGASKQKISPRDIEEIAAHSGLPVFVKGIQHPEDAD
MAIKAGASGIWVSNHGARQLYEAPGSFDTLPAIAERVNKRVPIVFDSGVR
RGEHVAKALASGADVVALGRPVLFGLALGGWQGAYSVLDYFQKDLTRVMQ
LTGSQNVEDLKGLDLFDNPYGYEY
A96L CC1 mutant
(SEQ ID NO: 20)
MNNNDIEYNAPSEIKYIDVCNTYDLEEEASKVVPHGGFNYIAGASGDEWT
KRANDRAWKHKLLYPRLAQDVEAPDTSTEILGHKIKAPFIMAPIALHGLA
HTTKEAGTARAVSEFGTIMSISAYSGATFEEISEGLNGGPRWFQIYMAKD
DQQNRDILDEAKSDGATAIILTADSTVSGNRDRDCKNKFVYPFGMPIVQR
YLRGTAEGMSLNNIYGASKQKISPRDIEEIAAHSGLPVFVKGIQHPEDAD
MAIKAGASGIWVSNHGARQLYEAPGSFDTLPAIAERVNKRVPIVFDSGVR
RGEHVAKALASGADVVALGRPVLFGLALGGWQGAYSVLDYFQKDLTRVMQ
LTGSQNVEDLKGLDLFDNPYGYEY
A96L/N212K CC1 mutant
(SEQ ID NO: 21)
MNNNDIEYNAPSEIKYIDVCNTYDLEEEASKVVPHGGFNYIAGASGDEWT
KRANDRAWKHKLLYPRLAQDVEAPDTSTEILGHKIKAPFIMAPIALHGLA
HTTKEAGTARAVSEFGTIMSISAYSGATFEEISEGLNGGPRWFQIYMAKD
DQQNRDILDEAKSDGATAIILTADSTVSGNRDRDCKNKFVYPFGMPIVQR
YLRGTAEGMSLKNIYGASKQKISPRDIEEIAAHSGLPVFVKGIQHPEDAD
MAIKAGASGIWVSNHGARQLYEAPGSFDTLPAIAERVNKRVPIVFDSGVR
RGEHVAKALASGADVVALGRPVLFGLALGGWQGAYSVLDYFQKDLTRVMQ
LTGSQNVEDLKGLDLFDNPYGYEY
A96L/N212K CC2 mutant
(SEQ ID NO: 22)
MNNNDIEYNAPSEIKYIDVVNTYDLEEEASKVVPHGGFNYIAGASGDEWT
KRANDRAWKHKLLYPRLAQDVEAPDTSTEILGHKIKAPFIMAPIALHGLA
HTTKEAGTARAVSEFGTIMSISAYSGATCEEISEGLNGGPRWFQIYMAKD
DQQNRDILDEAKSCGATAIILTADSTVSGNRDRDVKNKFVYPFGMPIVQR
YLRGTAEGMSLKNIYGASKQKISPRDIEEIAAHSGLPVFVKGIQHPEDAD
MAIKAGASGIWVSNHGARQLYEAPGSFDTLPAIAERVNKRVPIVFDSGVR
RGEHVAKALASGADVVALGRPVLFGLALGGWQGAYSVLDYFQKDLTRVMQ
LTGSQNVEDLKGLDLFDNPYGYEY
A96L/N212K CC3 mutant
(SEQ ID NO: 23)
MNNNDIEYNAPSEIKYIDVCNTYDLEEEASKVVPHGGFNYIAGASGDEWT
KRANDRAWKHKLLYPRLAQDVEAPDTSTEILGHKIKAPFIMAPIALHGLA
HTTKEAGTARAVSEFGTIMSISAYSGATCEEISEGLNGGPRWFQIYMAKD
DQQNRDILDEAKSCGATAIILTADSTVSGNRDRDCKNKFVYPFGMPIVQR
YLRGTAEGMSLKNIYGASKQKISPRDIEEIAAHSGLPVFVKGIQHPEDAD
MAIKAGASGIWVSNHGARQLYEAPGSFDTLPAIAERVNKRVPIVFDSGVR
RGEHVAKALASGADVVALGRPVLFGLALGGWQGAYSVLDYFQKDLTRVMQ
LTGSQNVEDLKGLDLFDNPYGYEY
b2LOxS mutant
(SEQ ID NO: 24)
MNDDERKVTPEELAKHNTGTDCWVAINGKVYDLTEFLPQHPGGRNVILKR
AGKDASKVFNPIHPPDAISKFLPADKFVGVLDGELPEEEEVLTNNNDIEY
NAPSEIKYIDVVNTYDLEEEASKVVPHGGFNYIAGASGDEWTKRANDRAW
KHKLLYPRLAQDVEAPDTSTEILGHKIKAPFIMAPISLHGLAHTTKEAGT
ARAVSEFGTIMSISAYSGATFEEISEGLNGGPRWFQIYMAKDDQQNRDIL
DEAKSDGATAIILTADSTVSGNRDRDVKNKFVYPFGMPIVQRYLRGTAEG
MSLKNIYGASKQKISPRDIEEIAAHSGLPVFVKGIQHPEDADMAIKAGAS
GIWVSNHGARQLYEAPGSFDTLPAIAERVNKRVPIVFDSGVRRGEHVAKA
LASGADVVALGRPVLFGLALGGWQGAYSVLDYFQKDLTRVMQLTGSQNVE
DLKGLDLFDNPYGYEY
b2LOx CC3 mutant
(SEQ ID NO: 25)
MNDDERKVTPEELAKHNTGTDCWVAINGKVYDLTEFLPQHPGGRNVILKR
AGKDASKVFNPIHPPDAISKFLPADKFVGVLDGELPEEEEVLTNNNDIEY
NAPSEIKYIDVCNTYDLEEEASKVVPHGGFNYIAGASGDEWTKRANDRAW
KHKLLYPRLAQDVEAPDTSTEILGHKIKAPFIMAPIALHGLAHTTKEAGT
ARAVSEFGTIMSISAYSGATCEEISEGLNGGPRWFQIYMAKDDQQNRDIL
DEAKSCGATAIILTADSTVSGNRDRDCKNKFVYPFGMPIVQRYLRGTAEG
MSLKNIYGASKQKISPRDIEEIAAHSGLPVFVKGIQHPEDADMAIKAGAS
GIWVSNHGARQLYEAPGSFDTLPAIAERVNKRVPIVFDSGVRRGEHVAKA
LASGADVVALGRPVLFGLALGGWQGAYSVLDYFQKDLTRVMQLTGSQNVE
DLKGLDLFDNPYGYEY

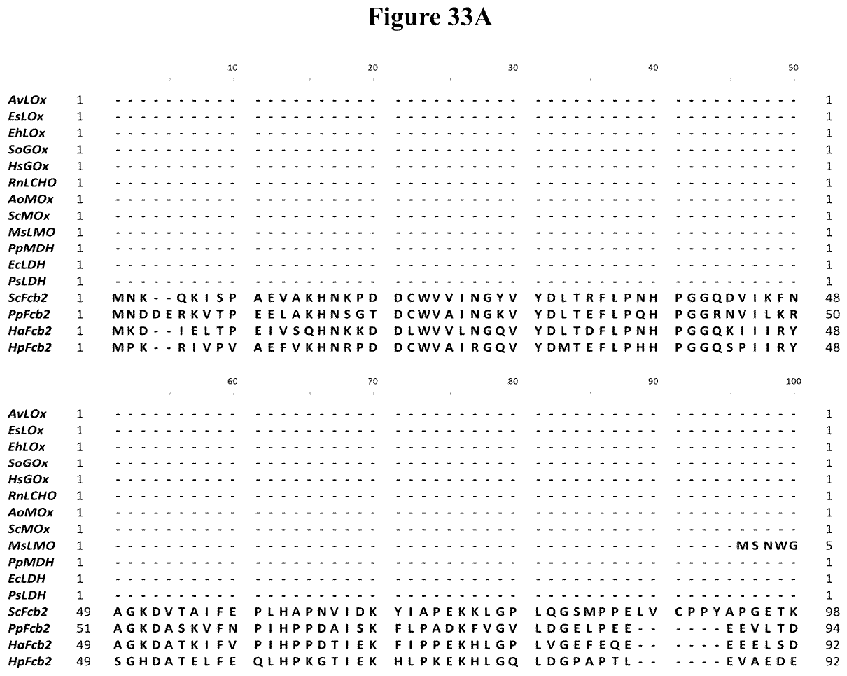

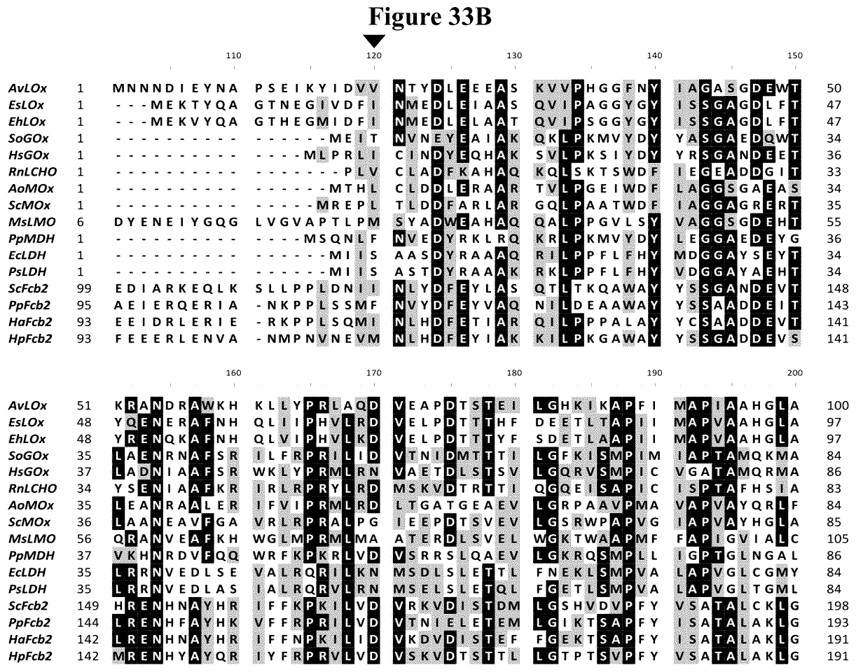

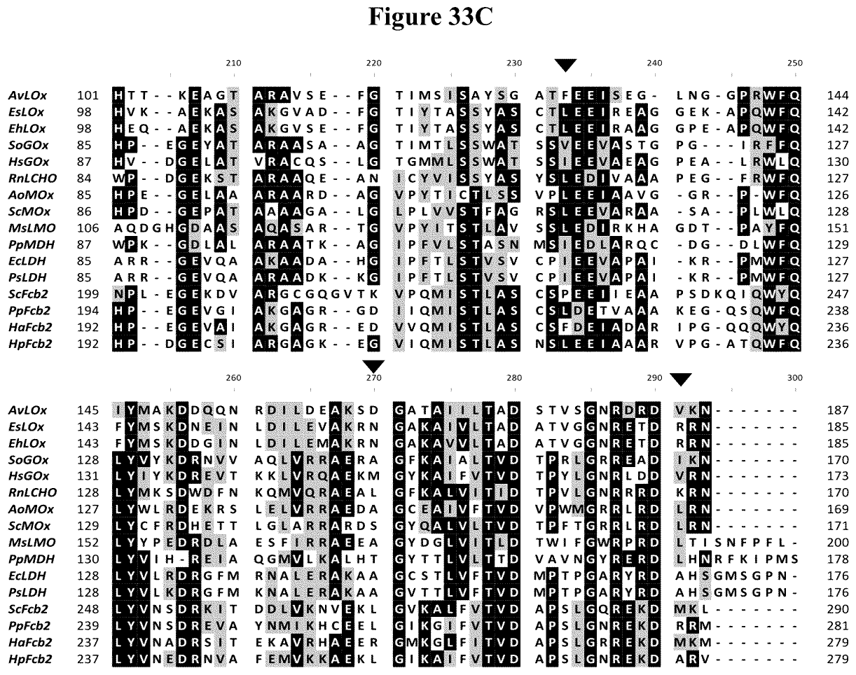

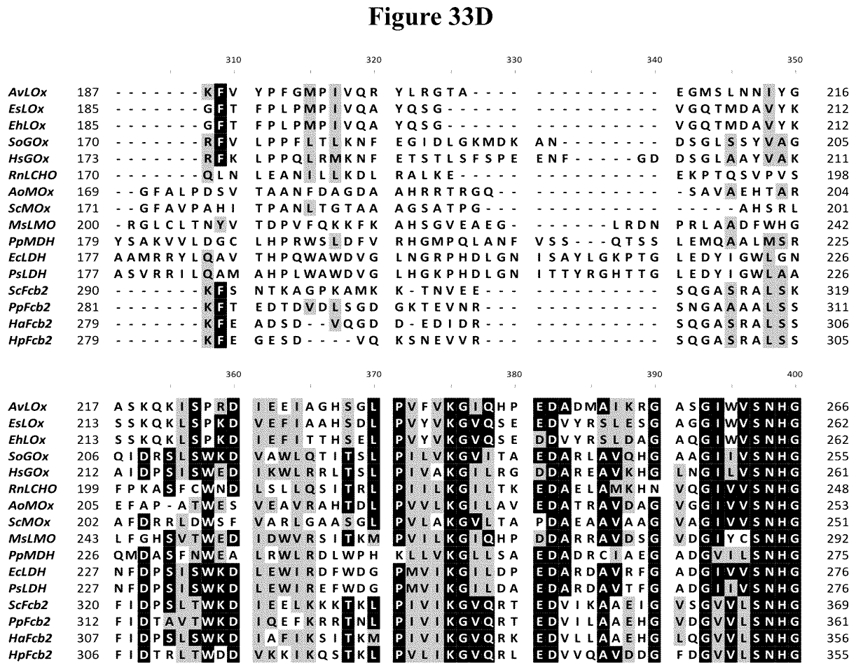

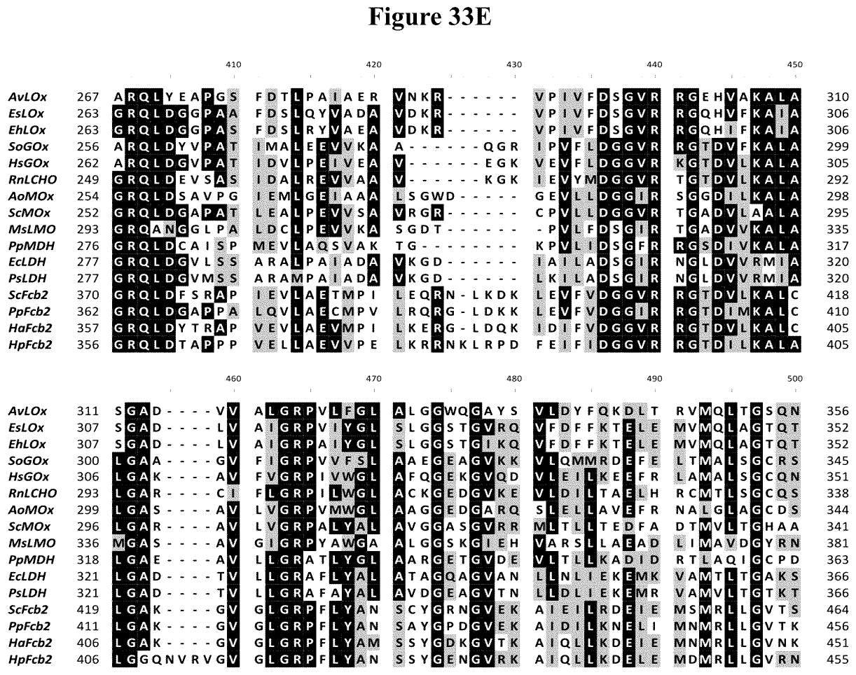

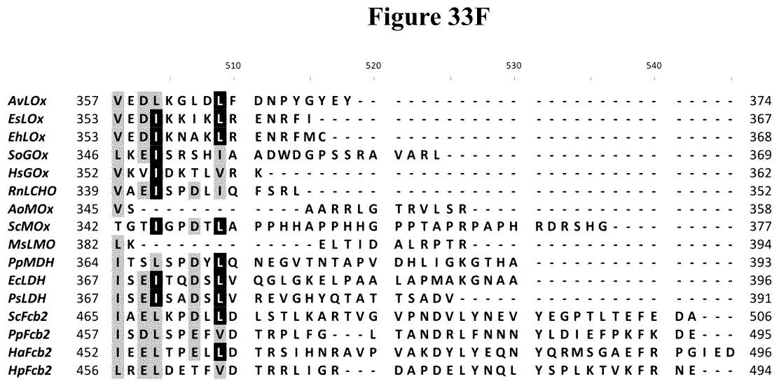

FIGS. 33A-E illustrate the amino acid sequence alignment of FMN dependent α-hydroxyacid oxidizing flavoprotein family members. Alignment was prepared using ClustalW, Graphic view was prepared by BioEdit software ver. 7.2.6. Arrows indicate candidates of cysteine mutation site. The aligned sequences include Aerococcus viridans LOx (AvLOx) (SEQ ID NO: 1), Enterococcus spp LOx (EsLOx) (SEQ ID NO: 2), Enterococcus hirae LOx (EhLOx) (SEQ ID NO: 3), Spinacia oleraceaglycolate oxidase (SoGOx) (SEQ ID NO: 4), Homo sapiens GOx (HsGOx) (SEQ ID NO: 11), Rattus norvegicus long-chain hydroxy acid oxidase (RnLCHO) (SEQ ID NO: 12), Amycolatopsis orientalis mandelate oxidase (AoMOx) (SEQ ID NO: 13), Streptomyces coelicolor MOx (ScMOx) (SEQ ID NO: 14), Mycobacterium smegmatis lactate monooxygenase (MsLMO) (SEQ ID NO: 15), Pseudomonas putida mandelate dehydrogenase (PpMDH) (SEQ ID NO: 16), Escherichia coli lactate dehydrogenase (EcLDH) (SEQ ID NO: 9), Pseudomonas stutzeri SDM lactate dehydrogenase (PsLDH) (SEQ ID NO: 10), Saccharomyces cerevisiae flavocytochrome b2 (ScFcb2) (SEQ ID NO: 6), Pichia pastoris Fcb2 (PpFcb2) (SEQ ID NO: 5), Hansenula anomala Fcb2 (HaFcb2) (SEQ ID NO:7), and Hansenula polymorphs Fcb2 (HpFcb2) (SEQ ID NO: 8). These Figures illustrate how amino acid positions in each of SEQ ID NOs: 2-16 correspond to positions in SEQ ID NO: 1.

Many modifications and other embodiments of the subject matter set forth herein will come to mind to one skilled in the art to which the subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation. Each embodiment disclosed herein is contemplated as being applicable to each of the other disclosed embodiments. All combinations and sub-combinations of the various elements described herein are within the scope of the embodiments.

What is claimed is:

1. An engineered lactate oxidoreductase with increased stability as compared to the wild-type.

2. The engineered lactate oxidoreductase of claim 1, comprising a sequence having at least 90% sequence identity to any one of SEQ ID NOs:1-16, provided at least one of the amino acids at a position in said sequence corresponding to positions 10 to 30, positions 119-139, positions 154-174, or positions 175 to 195 of SEQ ID NO:1 is different from the amino acid occupying the corresponding position in said SEQ ID NO: 1-16.

3. The engineered lactate oxidoreductase of claim 2, comprising a sequence having at least 90% sequence identity to SEQ ID NO:1 provided at least one of positions 10 to 30 or positions 175 to 195 is different from the amino acid occupying the corresponding position in SEQ ID NO: 1.

4. The engineered lactate oxidoreductase of claim 2 or 3, comprising a modification at one or more amino acid positions selected from:
(a) a position corresponding to position 20 of the amino acid sequence set forth in SEQ ID NO: 1,
(b) a position corresponding to position 185 of the amino acid sequence set forth in SEQ ID NO: 1,
(c) a position corresponding to position 129 of the amino acid sequence set forth in SEQ ID NO: 1, and
(d) a position corresponding to position 164 of the amino acid sequence set forth in SEQ ID NO: 1.

5. The engineered lactate oxidoreductase of claim 4, comprising a modification at one or more amino acid positions selected from:
(a) a position corresponding to position 20 of the amino acid sequence set forth in SEQ ID NO: 1 and
(b) a position corresponding to position 185 of the amino acid sequence set forth in SEQ ID NO: 1.

6. The engineered lactate oxidoreductase of claim 2 or 3, comprising a sequence having at least 90% sequence identity to SEQ ID NO:1, provided at least one of position 20, position 129, position 164, or position 185 is different from the amino acid occupying the corresponding position in SEQ ID NO: 1.

7. The engineered lactate oxidoreductase of claim 6, comprising a sequence having at least 90% sequence identity to SEQ ID NO:1, provided at least one of positions 20 or 185 is different from the amino acid occupying the corresponding position in SEQ ID NO: 1.

8. The engineered lactate oxidoreductase of claim 4, comprising a modification at one or more amino acid positions selected from:
(a) a position corresponding to position 20 of the amino acid sequence set forth in SEQ ID NO: 1, wherein the modification includes a substitution of the wild-type amino acid residue with an amino acid residue Cys;
(b) a position corresponding to position 185 of the amino acid sequence set forth in SEQ ID NO: 1, wherein the modification includes a substitution of the wild-type amino acid residue with an amino acid residue Cys;
(c) a position corresponding to position 129 of the amino acid sequence set forth in SEQ ID NO: 1, wherein the modification includes a substitution of the wild-type amino acid residue with an amino acid residue Cys; and
(d) a position corresponding to position 164 of the amino acid sequence set forth in SEQ ID NO: 1, wherein the modification includes a substitution of the wild-type amino acid residue with an amino acid residue Cys.

9. The engineered lactate oxidoreductase of claim 8, comprising a modification at one or more amino acid positions selected from:
(a) a position corresponding to position 20 of the amino acid sequence set forth in SEQ ID NO: 1, wherein the modification includes a substitution of the amino acid residue Val with an amino acid residue Cys; and
(b) a position corresponding to position 185 of the amino acid sequence set forth in SEQ ID NO: 1, wherein the modification includes a substitution of the amino acid residue Val with an amino acid residue Cys.

10. The engineered lactate oxidoreductase of any of the preceding claims, further comprising a modification at a position corresponding to position 96 of the amino acid sequence set forth in SEQ ID NO: 1, wherein the modification includes a substitution of the wild-type amino acid residue with an amino acid residue Leu.

11. The engineered lactate oxidoreductase of claim 10, wherein the wild-type amino acid residue at position 96 is Ala.

12. The engineered lactate oxidoreductase of any of the preceding claims further comprising a modification at a position corresponding to position 212 of the amino acid sequence set forth in SEQ ID NO: 1, wherein the modification includes a substitution of the wild-type amino acid residue with an amino acid residue Lys.

13. The engineered lactate oxidoreductase of claim 12, wherein the wild-type amino acid residue at position 212 is Asn.

14. The engineered lactate oxidoreductase of any of the preceding claims further comprising a modification at a position corresponding to position 95 of the amino acid sequence set forth in SEQ ID NO: 1, wherein the modification includes a substitution of the wild-type amino acid residue with an amino acid residue Ser.

15. The engineered lactate oxidoreductase of claim 14, wherein the wild-type amino acid residue at position 95 is Ala.

16. The engineered lactate oxidoreductase of any one of claims 10-13, which includes a reduced oxidase activity as compared to the wild-type lactate oxidoreductase.

17. The engineered lactate oxidoreductase of any one of claims 10-13, which includes an increased dehydrogenase activity compared to the wild-type lactate oxidoreductase.

18. The engineered lactate oxidoreductase of claim 14 or 15, which includes an increased Km.

19. The engineered lactate oxidoreductase of claim 8 or 9, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, and iii) a position corresponding to position 96 of the amino acid sequence set forth in SEQ ID NO: 1, wherein the modification includes a substitution of the wild-type amino acid residue with an amino acid residue Leu.

20. The engineered lactate oxidoreductase of claim 19, wherein the wild-type amino acid residue at position 20 is Val; the wild-type amino acid residue at position 185 is Val; and the wild-type amino acid residue at position 96 is Ala.

21. The engineered lactate oxidoreductase of claim 8 or 9, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, and iii) a position corresponding to position 212 of the amino acid sequence set forth in SEQ ID NO: 1, wherein the modification includes a substitution of the wild-type amino acid residue with an amino acid residue Lys.

22. The engineered lactate oxidoreductase of claim 21, wherein the wild-type amino acid residue at position 20 is Val; the wild-type amino acid residue at position 185 is Val; and the wild-type amino acid residue at position 212 is Asn.

23. The engineered lactate oxidoreductase of claim 8 or 9, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, and iii) a position corresponding to position 95 of the amino acid sequence set forth in SEQ ID NO: 1, wherein the modification includes a substitution of the wild-type amino acid residue with an amino acid residue Ser.

24. The engineered lactate oxidoreductase of claim 23, wherein the wild-type amino acid residue at position 20 is Val; the wild-type amino acid residue at position 185 is Val; and the wild-type amino acid residue at position 95 is Ala.

25. The engineered lactate oxidoreductase of claim 8 or 9, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, iii) a position corresponding to position 96 of the amino acid sequence set forth in SEQ ID NO: 1, wherein the modification includes a substitution of the wild-type amino acid residue with an amino acid residue Leu, and iv) a position corresponding to position 212 of the amino acid sequence set forth in SEQ ID NO: 1, wherein the modification includes a substitution of the wild-type amino acid residue with an amino acid residue Lys.

26. The engineered lactate oxidoreductase of claim 25, wherein the wild-type amino acid residue at position 20 is Val; the wild-type amino acid residue at position 185 is Val; the wild-type amino acid residue at position 96 is Ala; and the wild-type amino acid residue at position 212 is Asn.

27. The engineered lactate oxidoreductase of claim 8 or 9, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, iii) a position corresponding to position 96 of the amino acid sequence set forth in SEQ ID NO: 1, wherein the modification includes a substitution of the wild-type amino acid residue with an amino acid residue Leu, and iv) a position corresponding to position 95 of the amino acid sequence set forth in SEQ ID NO: 1, wherein the modification includes a substitution of the wild-type amino acid residue with an amino acid residue Ser.

28. The engineered lactate oxidoreductase of claim 27, wherein the wild-type amino acid residue at position 20 is Val; the wild-type amino acid residue at position 185 is Val; the wild-type amino acid residue at position 96 is Ala; and the wild-type amino acid residue at position 95 is Ala.

29. The engineered lactate oxidoreductase of claim 8 or 9, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, iii) a position corresponding to position 212 of the amino acid sequence set forth in SEQ ID NO: 1, wherein the modification includes a substitution of the wild-type amino acid residue with an amino acid residue Lys, and iv) a position corresponding to position 95 of the amino acid sequence set forth in SEQ ID NO: 1, wherein the modification includes a substitution of the wild-type amino acid residue with an amino acid residue Ser.

30. The engineered lactate oxidoreductase of claim 29, wherein the wild-type amino acid residue at position 20 is Val; the wild-type amino acid residue at position 185 is Val; the wild-type amino acid residue at position 212 is Asn; and the wild-type amino acid residue at position 95 is Ala.

31. The engineered lactate oxidoreductase of claim 8 or 9, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, iii) a position corresponding to position 96 of the amino acid sequence set forth in SEQ ID NO: 1, wherein the modification includes a substitution of the wild-type amino acid residue with an amino acid residue Leu, iv) a position corresponding to position 212 of the amino acid sequence set forth in SEQ ID NO: 1, wherein the modification includes a substitution of the wild-type amino acid residue with an amino acid residue Lys, and v) a position corresponding to position 95 of the amino acid sequence set forth in SEQ ID NO: 1, wherein the modification includes a substitution of the wild-type amino acid residue with an amino acid residue Ser.

32. The engineered lactate oxidoreductase of claim 31, wherein the wild-type amino acid residue at position 20 is Val; the wild-type amino acid residue at position 185 is Val; the wild-type amino acid residue at position 96 is Ala; the wild-type amino acid residue at position 212 is Asn; and the wild-type amino acid residue at position 95 is Ala.

33. The engineered lactate oxidoreductase of claim 8 or 9, comprising a modification at i) a position corresponding to position 129 of the amino acid sequence set forth in SEQ ID NO: 1, wherein the modification includes a substitution of the wild-type amino acid residue with an amino acid residue Cys, ii) a position corresponding to position 164 of the amino acid sequence set forth in SEQ ID NO: 1, wherein the modification includes a substitution of the wild-type amino acid residue with an amino acid residue Cys, and iii) a position corresponding to position 96 of the amino acid sequence set forth in SEQ ID NO: 1, wherein the modification includes a substitution of the wild-type amino acid residue with an amino acid residue Leu.

34. The engineered lactate oxidoreductase of claim 8 or 9, comprising a modification at i) a position corresponding to position 129 of the amino acid sequence set forth in SEQ ID NO: 1, wherein the modification includes a substitution of the wild-type amino acid residue with an amino acid residue Cys, ii) a position corresponding to position 164 of the amino acid sequence set forth in SEQ ID NO: 1, wherein the modification includes a substitution of the wild-type amino acid residue with an amino acid residue Cys, and iii) a position corresponding to position 212 of the amino acid sequence set forth in SEQ ID NO: 1, wherein the modification includes a substitution of the wild-type amino acid residue with an amino acid residue Lys.

35. The engineered lactate oxidoreductase of claim 8 or 9, comprising a modification at i) a position corresponding to position 129 of the amino acid sequence set forth in SEQ ID NO: 1, wherein the modification includes a substitution of the wild-type amino acid residue with an amino acid residue Cys, ii) a position corresponding to position 164 of the amino acid sequence set forth in SEQ ID NO: 1, wherein the modification includes a substitution of the wild-type amino acid residue with an amino acid residue Cys, and iii) a position corresponding to position 95 of the amino acid sequence set forth in SEQ ID NO: 1, wherein the modification includes a substitution of the wild-type amino acid residue with an amino acid residue Ser.

36. The engineered lactate oxidoreductase of claim 8 or 9, comprising a modification at i) a position corresponding to position 129 of the amino acid sequence set forth in SEQ ID NO: 1, wherein the modification includes a substitution of the wild-type amino acid residue with an amino acid residue Cys, ii) a position corresponding to position 164 of the amino acid sequence set forth in SEQ ID NO: 1, wherein the modification includes a substitution of the wild-type amino acid residue with an amino acid residue Cys, iii) a position corresponding to position 96 of the amino acid sequence set forth in SEQ ID NO: 1, wherein the modification includes a substitution of the wild-type amino acid residue with an amino acid residue Leu, and iv) a position corresponding to position 212 of the amino acid sequence set forth in SEQ ID NO: 1, wherein the modification includes a substitution of the wild-type amino acid residue with an amino acid residue Lys.

37. The engineered lactate oxidoreductase of claim 8 or 9, comprising a modification at i) a position corresponding to position 129 of the amino acid sequence set forth in SEQ ID NO: 1, wherein the modification includes a substitution of the wild-type amino acid residue with an amino acid residue Cys, ii) a position corresponding to position 164 of the amino acid sequence set forth in SEQ ID NO: 1, wherein the modification includes a substitution of the wild-type amino acid residue with an amino acid residue Cys, iii) a position corresponding to position 96 of the amino acid sequence set forth in SEQ ID NO: 1, wherein the modification includes a substitution of the wild-type amino acid residue with an amino acid residue Leu, and iv) a position corresponding to position 95 of the amino acid sequence set forth in SEQ ID NO: 1, wherein the modification includes a substitution of the wild-type amino acid residue with an amino acid residue Ser.

38. The engineered lactate oxidoreductase of claim 8 or 9, comprising a modification at i) a position corresponding to position 129 of the amino acid sequence set forth in SEQ ID NO: 1, wherein the modification includes a substitution of the wild-type amino acid residue with an amino acid residue Cys, ii) a position corresponding to position 164 of the amino acid sequence set forth in SEQ ID NO: 1, wherein the modification includes a substitution of the wild-type amino acid residue with an amino acid residue Cys, iii) a position corresponding to position 212 of the amino acid sequence set forth in SEQ ID NO: 1, wherein the modification includes a substitution of the wild-type amino acid residue with an amino acid residue Lys, and iv) a position corresponding to position 95 of the amino acid sequence set forth in SEQ ID NO: 1, wherein the modification includes a substitution of the wild-type amino acid residue with an amino acid residue Ser.

39. The engineered lactate oxidoreductase of claim 8 or 9, comprising a modification at i) a position corresponding to position 129 of the amino acid sequence set forth in SEQ ID NO: 1, wherein the modification includes a substitution of the wild-type amino acid residue with an amino acid residue Cys, ii) a position corresponding to position 164 of the amino acid sequence set forth in SEQ ID NO: 1, wherein the modification includes a substitution of the wild-type amino acid residue with an amino acid residue Cys, iii) a position corresponding to position 96 of the amino acid sequence set forth in SEQ ID NO: 1, wherein the modification includes a substitution of the wild-type amino acid residue with an amino acid residue Leu, iv) a position corresponding to position 212 of the amino acid sequence set forth in SEQ ID NO: 1, wherein the modification includes a substitution of the wild-type amino acid residue with an amino acid residue Lys, and v) a position corresponding to position 95 of the amino acid sequence set forth in SEQ ID NO: 1, wherein the modification includes a substitution of the wild-type amino acid residue with an amino acid residue Ser.

40. The engineered lactate oxidoreductase of claim 8 or 9, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, iii) a position corresponding to position 129 of the amino acid sequence set forth in SEQ ID NO: 1, wherein the modification includes a substitution of the wild-type amino acid residue with an amino acid residue Cys, iv) a position corresponding to position 164 of the amino acid sequence set forth in SEQ ID NO: 1, wherein the modification includes a substitution of the wild-type amino acid residue with an amino acid residue Cys, and v) a position corresponding to position 96 of the amino acid sequence set forth in SEQ ID NO: 1, wherein the modification includes a substitution of the wild-type amino acid residue with an amino acid residue Leu.

41. The engineered lactate oxidoreductase of claim 8 or 9, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, iii) a position corresponding to position 129 of the amino acid sequence set forth in SEQ ID NO: 1, wherein the modification includes a substitution of the wild-type amino acid residue with an amino acid residue Cys, iv) a position corresponding to position 164 of the amino acid sequence set forth in SEQ ID NO: 1, wherein the modification includes a substitution of the wild-type amino acid residue with an amino acid residue Cys, and v) a position corresponding to position 212 of the amino acid sequence set forth in SEQ ID NO: 1, wherein the modification includes a substitution of the wild-type amino acid residue with an amino acid residue Lys.

42. The engineered lactate oxidoreductase of claim 8 or 9, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, iii) a position corresponding to position 129 of the amino acid sequence set forth in SEQ ID NO: 1, wherein the modification includes a substitution of the wild-type amino acid residue with an amino acid residue Cys, iv) a position corresponding to position 164 of the amino acid sequence set forth in SEQ ID NO: 1, wherein the modification includes a substitution of the wild-type amino acid residue with an amino acid residue Cys, and v) a position corresponding to position 95 of the amino acid sequence set forth in SEQ ID NO: 1, wherein the modification includes a substitution of the wild-type amino acid residue with an amino acid residue Ser.

43. The engineered lactate oxidoreductase of claim 8 or 9, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, iii) a position corresponding to position 129 of the amino acid sequence set forth in SEQ ID NO: 1, wherein the modification includes a substitution of the wild-type amino acid residue with an amino acid residue Cys, iv) a position corresponding to position 164 of the amino acid sequence set forth in SEQ ID NO: 1, wherein the modification includes a substitution of the wild-type amino acid residue with an amino acid residue Cys, v) a position corresponding to position 96 of the amino acid sequence set forth in SEQ ID NO: 1, wherein the modification includes a substitution of the wild-type amino acid residue with an amino acid residue Leu, and vi) a position corresponding to position 212 of the amino acid sequence set forth in SEQ ID NO: 1, wherein the modification includes a substitution of the wild-type amino acid residue with an amino acid residue Lys.

44. The engineered lactate oxidoreductase of claim 8 or 9, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, iii) a position corresponding to position 129 of the amino acid sequence set forth in SEQ ID NO: 1, wherein the modification includes a substitution of the wild-type amino acid residue with an amino acid residue Cys, iv) a position corresponding to position 164 of the amino acid sequence set forth in SEQ ID NO: 1, wherein the modification includes a substitution of the wild-type amino acid residue with an amino acid residue Cys, v) a position corresponding to position 96 of the amino acid sequence set forth in SEQ ID NO: 1, wherein the modification includes a substitution of the wild-type amino acid residue with an amino acid residue Leu, and vi) a position corresponding to position 95 of the amino acid sequence set forth in SEQ ID NO: 1, wherein the modification includes a substitution of the wild-type amino acid residue with an amino acid residue Ser.

45. The engineered lactate oxidoreductase of claim 8 or 9, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, iii) a position corresponding to position 129 of the amino acid sequence set forth in SEQ ID NO: 1, wherein the modification includes a substitution of the wild-type amino acid residue with an amino acid residue Cys, iv) a position corresponding to position 164 of the amino acid sequence set forth in SEQ ID NO: 1, wherein the modification includes a substitution of the wild-type amino acid residue with an amino acid residue Cys, v) a position corresponding to position 212 of the amino acid sequence set forth in SEQ ID NO: 1, wherein the modification includes a substitution of the wild-type amino acid residue with an amino acid residue Lys, and vi) a position corresponding to position 95 of the amino acid sequence set forth in SEQ ID NO: 1, wherein the modification includes a substitution of the wild-type amino acid residue with an amino acid residue Ser.

46. The engineered lactate oxidoreductase of claim 8 or 9, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, iii) a position corresponding to position 129 of the amino acid sequence set forth in SEQ ID NO: 1, wherein the modification includes a substitution of the wild-type amino acid residue with an amino acid residue Cys, iv) a position corresponding to position 164 of the amino acid sequence set forth in SEQ ID NO: 1, wherein the modification includes a substitution of the wild-type amino acid residue with an amino acid residue Cys, v) a position corresponding to position 96 of the amino acid sequence set forth in SEQ ID NO: 1, wherein the modification includes a substitution of the wild-type amino acid residue with an amino acid residue Leu, vi) a position corresponding to position 212 of the amino acid sequence set forth in SEQ ID NO: 1, wherein the modification includes a substitution of the wild-type amino acid residue with an amino acid residue Lys, and vii) a position corresponding to position 95 of the amino acid sequence set forth in SEQ ID NO: 1, wherein the modification includes a substitution of the wild-type amino acid residue with an amino acid residue Ser.

47. The engineered lactate oxidoreductase of any of the preceding claims, further comprising a fused cytochrome domain of flavocytochrome b2.

48. An engineered lactate oxidoreductase comprising a fused cytochrome domain of flavocytochrome b2.

49. An engineered lactate oxidoreductase comprising a modification at a position corresponding to position 95 of the amino acid sequence set forth in SEQ ID NO: 1, wherein the modification includes a substitution of the wild-type amino acid residue with an amino acid residue Ser.

50. The engineered lactate oxidoreductase of claim 49, wherein the wild-type amino acid residue at position 95 is Ala.

51. The engineered lactate oxidoreductase of claim 1, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, iii) a position corresponding to position 96 of the amino acid sequence set forth in SEQ ID NO: 1, wherein the modification includes a substitution of the wild-type amino acid residue with an amino acid residue Leu, iv) a position corresponding to position 212 of the amino acid sequence set forth in SEQ ID NO: 1, wherein the modification includes a substitution of the wild-type amino acid residue with an amino acid residue Lys, and v) a position corresponding to position 95 of the amino acid sequence set forth in SEQ ID NO: 1, wherein the modification includes a substitution of the wild-type amino acid residue with an amino acid residue Ser, and fused to a cytochrome domain of flavocytochrome b2.

52. The engineered lactate oxidoreductase of claim 51, wherein the wild-type amino acid residue at position 20 is Val; the wild-type amino acid residue at position 185 is Val; the wild-type amino acid residue at position 96 is Ala; the wild-type amino acid residue at position 212 is Asn; and the wild-type amino acid residue at position 95 is Ala.

53. The engineered lactate oxidoreductase of claim 1, comprising a modification at i) a position corresponding to position 129 of the amino acid sequence set forth in SEQ ID NO: 1, wherein the modification includes a substitution of the wild-type amino acid residue with an amino acid residue Cys, ii) a position corresponding to position 164 of the amino acid sequence set forth in SEQ ID NO: 1, wherein the modification includes a substitution of the wild-type amino acid residue with an amino acid residue Cys, iii) a position corresponding to position 96 of the amino acid sequence set forth in SEQ ID NO: 1, wherein the modification includes a substitution of the wild-type amino acid residue with an amino acid residue Leu, iv) a position corresponding to position 212 of the amino acid sequence set forth in SEQ ID NO: 1, wherein the modification includes a substitution of the wild-type amino acid residue with an amino acid residue Lys, and v) a position corresponding to position 95 of the amino acid sequence set forth in SEQ ID NO: 1, wherein the modification includes a substitution of the wild-type amino acid residue with an amino acid residue Ser, and fused to a cytochrome domain of flavocytochrome b2.

54. The engineered lactate oxidoreductase of claim 1, comprising a modification at i) a position corresponding to position 20 of the amino acid sequence set forth in SEQ ID NO: 1, wherein the modification includes a substitution of the wild-type amino acid residue with an amino acid residue Cys, ii) a position corresponding to position 185 of the amino acid sequence set forth in SEQ ID NO: 1, wherein the modification includes a substitution of the wild-type amino acid residue with an amino acid residue Cys, iii) a position corresponding to position 129 of the amino acid sequence set forth in SEQ ID NO: 1, wherein the modification includes a substitution of the wild-type amino acid residue with an amino acid residue Cys, iv) a position corresponding to position 164 of the amino acid sequence set forth in SEQ ID NO: 1, wherein the modification includes a substitution of the wild-type amino acid residue with an amino acid residue Cys, v) a position corresponding to position 96 of the amino acid sequence set forth in SEQ ID NO: 1, wherein the modification includes a substitution of the wild-type amino acid residue with an amino acid residue Leu, vi) a position corresponding to position 212 of the amino acid sequence set forth in SEQ ID NO: 1, wherein the modification includes a substitution of the wild-type amino acid residue with an amino acid residue Lys, and vii) a position corresponding to position 95 of the amino acid sequence set forth in SEQ ID NO: 1, wherein the modification includes a substitution of the wild-type amino acid residue with an amino acid residue Ser, and fused to a cytochrome domain of flavocytochrome b2.

55. The engineered lactate oxidoreductase of any of the preceeding claims, wherein the enzyme is selected from the group consisting of lactase oxidases, glycolate oxidases, long-chain hydroxy acid oxidases, mandelate oxidases, lactate monooxygenases, mandelate dehydrogenases, and lactate dehydrogenases.

56. A method of assaying lactate in a sample, the method comprising the steps of: contacting the sample with an engineered lactate oxidoreductase modified at one or more amino acid positions selected from: (i) a position corresponding to position 20 of the amino acid sequence set forth in SEQ ID NO: 1, wherein the modification includes a substitution of the wild-type amino acid residue with an amino acid residue Cys, (ii) a position corresponding to position 185 of the amino acid sequence set forth in SEQ ID NO: 1, wherein the modification includes a substitution of the wild-type amino acid residue with an amino acid residue Cys, (iii) a position corresponding to position 129 of the amino acid sequence set forth in SEQ ID NO: 1, wherein the modification includes a substitution of the wild-type amino acid residue with an amino acid residue Cys, and (iv) a position corresponding to position 164 of the amino acid sequence set forth in SEQ ID NO: 1, wherein the modification includes a substitution of the wild-type amino acid residue with an amino acid residue Cys; and measuring an amount of the lactate oxidized by the engineered lactate oxidoreductase.

57. The method of claim 56, wherein the engineered lactate oxidoreductase is modified at one or more amino acid positions selected from (i) a position corresponding to position 20 of the amino acid sequence set forth in SEQ ID NO: 1, wherein the modification includes a substitution of the amino acid residue Val with an amino acid residue Cys, and (ii) a position corresponding to position 185 of the amino acid sequence set forth in SEQ ID NO: 1, wherein the modification includes a substitution of the amino acid residue Val with an amino acid residue Cys.

58. A method of assaying lactate in a sample, the method comprising the steps of: contacting the sample with the engineered lactate oxidoreductase of claim 1; and measuring an amount of oxidized lactate.

59. The method of any one of claims 56-58, wherein the measurement is a continuous measurement.

60. A device for assaying lactate in a sample, the device comprising: the engineered lactate oxidoreductase of claim 1; and an electron mediator.

61. A kit for assaying lactate in a sample, the kit comprising: the engineered lactate oxidoreductase of claim 1; and an electron mediator.

62. An enzyme electrode comprising the engineered lactate oxidoreductase of claim 1 immobilized on an electrode.

63. An enzyme sensor for assaying lactate comprising the enzyme electrode of claim 62 as a working electrode.

64. The engineered lactate oxidoreductase of claim 1, wherein the enzyme is a homotetramer comprising intra-subunit disulfide bonds.


SEQUENCE LISTING (25 sequences)

SEQ ID NO: 1 - protein, 374 aa, aerococcus viridans
MNNNDIEYNAPSEIKYIDVVNTYDLEEEASKVVPHGGFNYIAGASGDEWTKRANDRAWKHKLLYPRLAQDVEAPDTSTEILGHKIKAPFIMAPIAAHGLAHTTKEAGTARAVSEFGTIMSISAYSGATFEEISEGLNGGPRWFQIYMAKDDQQNRDILDEAKSDGATAIILTADSTVSGNRDRDVKNKFVYPFGMPIVQRYLRGTAEGMSLNNIYGASKQKISPRDIEEIAAHSGLPVFVKGIQHPEDADMAIKAGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY

SEQ ID NO: 2 - protein, 368 aa, Enterococcus hirae
MEKVYQAGTHEGMIDFINMEDLELAATQVIPSGGYGYISSGAGDLFTYRENQKAFNHQLVIPHVLKDVELPDTTTYFSDETLAAPIIMAPVAAHGLAHEQAEKASAKGVSEFGTIYTASSYASCTLEEIRAAGGPEAPQWFQFYMSKDDGINLDILEMAKRNGAKAVVLTADATVGGNRETDRRNGFTFPLPMPIVQAYQSGVGQTMDAVYKSSKQKLSPKDIEFITTHSELPVYVKGVQSEDDVYRSLDAGAQGIWVSNHGGRQLDGGPASFDSLRYVAEAVDKRVPIVFDSGVRRGQHIFKAIASGADLVAIGRPAIYGLSLGGSTGIKQVFDFFKTELEMVMQLAGTQTVEDIKNAKLRENRFMC

SEQ ID NO: 3 - protein, 367 aa, Enterococcus spp
MEKTYQAGTNEGIVDFINMEDLEIAASQVIPAGGYGYISSGAGDLFTYQENERAFNHQLIIPHVLRDVELPDTTTHFDEETLTAPIIMAPVAAHGLAHVKAEKASAKGVADFGTIYTASSYASCTLEEIREAGGEKAPQWFQFYMSKDNEINLDILEVAKRNGAKAIVLTADATVGGNRETDRRNGFTFPLPMPIVQAYQSGVGQTMDAVYKSSKQKLSPKDVEFIAAHSDLPVYVKGVQSEEDVYRSLESGAGGIWVSNHGGRQLDGGPAAFDSLQYVADAVDKRVPIVFDSGVRRGQHVFKAIASGADLVAIGRPVIYGLSLGGSTGVRQVFDFFKTELEMVMQLAGTQTVEDIKKIKLRENRFI

SEQ ID NO: 4 - protein, 369 aa, Spinacia oleracea
MEITNVNEYEAIAKQKLPKMVYDYYASGAEDQWTLAENRNAFSRILFRPRILIDVTNIDMTTTILGFKISMPIMIAPTAMQKMAHPEGEYATARAASAAGTIMTLSSWATSSVEEVASTGPGIRFFQLYVYKDRNVVAQLVRRAERAGFKAIALTVDTPRLGRREADIKNRFVLPPFLTLKNFEGIDLGKMDKANDSGLSSYVAGQIDRSLSWKDVAWLQTITSLPILVKGVITAEDARLAVQHGAAGIIVSNHGARQLDYVPATIMALEEVVKAAQGRIPVFLDGGVRRGTDVFKALALGAAGVFIGRPVVFSLAAEGEAGVKKVLQMMRDEFELTMALSGCRSLKEISRSHIAADWDGPSSRAVARL

SEQ ID NO: 5 - protein, 495 aa, Pichia pastoris
MNDDERKVTPEELAKHNTGTDCWVAINGKVYDLTEFLPQHPGGRNVILKRAGKDASKVFNPIHPPDAISKFLPADKFVGVLDGELPEEEEVLTDAEIERQERIANKPPLSSMFNVYDFEYVAQNILDEAAWAYYSSAADDEITLRENHFAYHKVFFRPRILVDVTNIELETEMLGIKTSAPFYISATALAKLGHPEGEVGIAKGAGRGDIIQMISTLASCSLDETVAAAKEGQSQWFQLYVNSDREVAYNMIKHCEELGIKGIFVTVDAPSLGNREKDRRMKFTEDTDVDLSGDGKTEVNRSNGAAAALSSFIDTAVTWKDIAEFKRRTNLPIVIKGIQRTEDVILAAEHGVDGVVLSNHGGRQLDGAPPSLQVLAECMPVLRQRGLDKKLEVFVDGGIRRGTDIMKALCLGAKGVGLGRPFLYANSAYGPDGVEKAIDILKNELIMNMRLLGVTKISDLSPEFVDTRPLFGLTANDRLFNNNYLDIEFPKFKDE

SEQ ID NO: 6 - protein, 506 aa, Saccharomyces cerevisiae
MNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPKILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEEAIEILRDEIEMSMRLLGVTSIAELKPDLLDLSTLKARTVGVPNDVLYNEVYEGPTLTEFEDA

SEQ ID NO: 7 - protein, 496 aa, Hansenula anomala
MKDIELTPEIVSQHNKKDDLWVVLNGQVYDLTDFLPNHPGGQKIIIRYAGKDATKIFVPIHPPDTIEKFIPPEKHLGPLVGEFEQEEEELSDEEIDRLERIERKPPLSQMINLHDFETIARQILPPPALAYYCSAADDEVTLRENHNAYHRIFFNPKILIDVKDVDISTEFFGEKTSAPFYISATALAKLGHPEGEVAIAKGAGREDVVQMISTLASCSFDEIADARIPGQQQWYQLYVNADRSITEKAVRHAEERGMKGLFITVDAPSLGRREKDMKMKFEADSDVQGDDGDIDRSQGASRALSSFIDPSLSWKDIAFIKSITKMPIVIKGVQRKEDVLLAAEHGLQGVVLSNHGGRQLDYTRAPVEVLAEVMPILKERGLDQKIDIFVDGGVRRGTDVLKALCLGAKGVGLGRPFLYAMSSYGDKGVTKAIQLLKDEIEMNMRLLGVNKIEELTPELLDTRSIHNRAVPVAKDYLYEQNYQRMSGAEFRPGIED

SEQ ID NO: 8 - protein, 494 aa, Hansenula polymorpha
MPKRIVPVDEFVKHNRPDDCWVAIRGQVYDMTEFLPQHPGGQSPIIRYSGHDATELFEQLHPKGTIEKNLPKDKHLGQLDGPAPTLEVAEDEFEEERLENVANMPNVNEVMNLHDFEYIAKKILPKGAWAYYSSGADDEVSMRENHYAYQRIYFRPRVLVDVSKVDTSTTLLGTPTSVPFYVSATALAKLGHPDGECSIARGAGKEGVIQMISTLASNSLEEIAAARVPGATQWFQLYVNEDRNVAFEMVKKAERLGIKAIFVTVDAPSLGNREKDARVKFEGESDVQKSNEVVRSQGASRALSSFIDTRLTWDDVIKIKQSTKLPVLIKGVQRLEDVVRAVDDGFDGVVLSNHGGRQLDTAPPPVELLAEVVPELRRRNKLRPDFEIFIDGGVRRGTDILKALALGGQNVRVGVGLGRPFLYANSAYGENGVRKAIQLLKDELEMDMRLLGVRNLRELDETFVDTRRLIGRDAPDELYNQLYSPLKTVKFRNE

SEQ ID NO: 9 - protein, 396 aa, Escherichia coli [sequence omitted]

SEQ ID NO: 10 - protein, 391 aa, Pseudomonas stutzeri
MIISASTDYRAAAKRKLPPFLFHYVDGGAYAEHTLRRNVEDLASIALRQRVLRNMSELSLETQLFGETLSMPVALAPVGLTGMLARRGEVQAARAADKKGIPFTLSTVSVCPIEEVAPAIKRPMWFQLYVLKDRGFMKNALERAKAAGVTTLVFTVDMPTPGARYRDAHSGMSGPNASVRRILQAMAHPLWAWDVGLHGKPHDLGNITTYRGHTTGLEDYIGWLAANFDPSISWKDLEWIREFWDGPMVIKGILDAEDARDAVTFGADGIIVSNHGGRQLDGVMSSARAMPAIADAVKGDLKILADSGIRNGLDVVRMIALGADTVLLGRAFAYALAVDGEAGVTNLLDLIEKEMRVAMVLTGTKTISEISADSLVREVGHYQTATTSADV

SEQ ID NO: 11 - protein, 362 aa, homo sapiens
MLPRLICINDYEQHAKSVLPKSIYDYYRSGANDEETLADNIAAFSRWKLYPRMLRNVAETDLSTSVLGQRVSMPICVGATAMQRMAHVDGELATVRACQSLGTGMMLSSWATSSIEEVAEAGPEALRWLQLYIYKDREVTKKLVRQAEKMGYKAIFVTVDTPYLGNRLDDVRNRFKLPPQLRMKNFETSTLSFSPEENFGDDSGLAAYVAKAIDPSISWEDIKWLRRLTSLPIVAKGILRGDDAREAVKHGLNGILVSNHGARQLDGVPATIDVLPEIVEAVEGKVEVFLDGGVRKGTDVLKALALGAKAVFVGRPIVWGLAFQGEKGVQDVLEILKEEFRLAMALSGCQNVKVIDKTLVRK

SEQ ID NO: 12 - protein, 352 aa, rattus norvegicus
PLVCLADFKAHAQKQLSKTSWDFIEGEADDGITYSENIAAFKRIRLRPRYLRDMSKVDTRTTIQGQEISAPICISPTAFHSIAWPDGEKSTARAAQEANICYVISSYASYSLEDIVAAAPEGFRWFQLYMKSDWDFNKQMVQRAEALGFKALVITIDTPVLGNRRRDKRNQLNLEANILLKDLRALKEEKPTQSVPVSFPKASFCWNDLSLLQSITRLPIILKGILTKEDAELAMKHNVQGIVVSNHGGRQLDEVSASIDALREVVAAVKGKIEVYMDGGVRTGTDVLKALALGARCIFLGRPILWGLACKGEDGVKEVLDILTAELHRCMTLSGCQSVAEISPDLIQFSRL

SEQ ID NO: 13 - protein, 358 aa, Amycolatopsis orientalis
MTHLCLDDLERAARTVLPGEIWDFLAGGSGAEASLEANRAALERIFVIPRMLRDLTGATGEAEVLGRPAAVPMAVAPVAYQRLFHPEGELAAARAARDAGVPYTICTLSSVPLEEIAAVGGRPWFQLYWLRDEKRSLELVRRAEDAGCEAIVFTVDVPWMGRRLRDLRNGFALPDSVTAANFDAGDAAHRRTRGQSAVAEHTAREFAPATWESVEAVRAHTDLPVVLKGILAVEDATRAVDAGVGGIVVSNHGGRQLDSAVPGIEMLGEIAAALSGWDGEVLLDGGIRSGGDILKALALGASAVLVGRPVMWGLAAGGEDGARQSLELLAVEFRNALGLAGCDSVSAARRLGTRVLSR

SEQ ID NO: 14 - protein, 377 aa, Streptomyces coelicolor
MREPLTLDDFARLARGQLPAATWDFIAGGAGRERTLAANEAVFGAVRLRPRALPGIEEPDTSVEVLGSRWPAPVGIAPVAYHGLAHPDGEPATAAAAGALGLPLVVSTFAGRSLEEVARAASAPLWLQLYCFRDHETTLGLARRARDSGYQALVLTVDTPFTGRRLRDLRNGFAVPAHITPANLTGTAAAGSATPGAHSRLAFDRRLDWSFVARLGAASGLPVLAKGVLTAPDAEAAVAAGVAGIVVSNHGGRQLDGAPATLEALPEVVSAVRGRCPVLLDGGVRTGADVLAALALGARAVLVGRPALYALAVGGASGVRRMLTLLTEDFADTMVLTGHAATGTIGPDTLAPPHHAPPHHGPPTAPRPAPHRDRSHG

SEQ ID NO: 15 - protein, 394 aa, Mycobacterium smegmatis
MSNWGDYENEIYGQGLVGVAPTLPMSYADWEAHAQQALPPGVLSYVAGGSGDEHTQRANVEAFKHWGLMPRMLMAATERDLSVELWGKTWAAPMFFAPIGVIALCAQDGHGDAASAQASARTGVPYITSTLAVSSLEDIRKHAGDTPAYFQLYYPEDRDLAESFIRRAEEAGYDGLVITLDTWIFGWRPRDLTISNFPFLRGLCLTNYVTDPVFQKKFKAHSGVEAEGLRDNPRLAADFWHGLFGHSVTWEDIDWVRSITKMPVILKGIQHPDDARRAVDSGVDGIYCSNHGGRQANGGLPALDCLPEVVKASGDTPVLFDSGIRTGADVVKALAMGASAVGIGRPYAWGAALGGSKGIEHVARSLLAEADLIMAVDGYRNLKELTIDALRPTR

SEQ ID NO: 16 - protein, 393 aa, Pseudomonas putida
MSQNLFNVEDYRKLRQKRLPKMVYDYLEGGAEDEYGVKHNRDVFQQWRFKPKRLVDVSRRSLQAEVLGKRQSMPLLIGPTGLNGALWPKGDLALARAATKAGIPFVLSTASNMSIEDLARQCDGDLWFQLYVIHREIAQGMVLKALHTGYTTLVLTTDVAVNGYRERDLHNRFKIPMSYSAKVVLDGCLHPRWSLDFVRHGMPQLANFVSSQTSSLEMQAALMSRQMDASFNWEALRWLRDLWPHKLLVKGLLSAEDADRCIAEGADGVILSNHGGRQLDCAISPMEVLAQSVAKTGKPVLIDSGFRRGSDIVKALALGAEAVLLGRATLYGLAARGETGVDEVLTLLKADIDRTLAQIGCPDITSLSPDYLQNEGVTNTAPVDHLIGKGTHA

SEQ ID NO: 17 - protein, 374 aa, aerococcus viridans
MNNNDIEYNAPSEIKYIDVVNTYDLEEEASKVVPHGGFNYIAGASGDEWTKRANDRAWKHKLLYPRLAQDVEAPDTSTEILGHKIKAPFIMAPIALHGLAHTTKEAGTARAVSEFGTIMSISAYSGATFEEISEGLNGGPRWFQIYMAKDDQQNRDILDEAKSDGATAIILTADSTVSGNRDRDVKNKFVYPFGMPIVQRYLRGTAEGMSLNNIYGASKQKISPRDIEEIAAHSGLPVFVKGIQHPEDADMAIKAGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY

SEQ ID NO: 18 - protein, 374 aa, aerococcus viridans
MNNNDIEYNAPSEIKYIDVVNTYDLEEEASKVVPHGGFNYIAGASGDEWTKRANDRAWKHKLLYPRLAQDVEAPDTSTEILGHKIKAPFIMAPIALHGLAHTTKEAGTARAVSEFGTIMSISAYSGATFEEISEGLNGGPRWFQIYMAKDDQQNRDILDEAKSDGATAIILTADSTVSGNRDRDVKNKFVYPFGMPIVQRYLRGTAEGMSLKNIYGASKQKISPRDIEEIAAHSGLPVFVKGIQHPEDADMAIKAGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY

SEQ ID NO: 19 - protein, 374 aa, aerococcus viridans
MNNNDIEYNAPSEIKYIDVVNTYDLEEEASKVVPHGGFNYIAGASGDEWTKRANDRAWKHKLLYPRLAQDVEAPDTSTEILGHKIKAPFIMAPISLHGLAHTTKEAGTARAVSEFGTIMSISAYSGATFEEISEGLNGGPRWFQIYMAKDDQQNRDILDEAKSDGATAIILTADSTVSGNRDRDVKNKFVYPFGMPIVQRYLRGTAEGMSLKNIYGASKQKISPRDIEEIAAHSGLPVFVKGIQHPEDADMAIKAGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY

SEQ ID NO: 20 - protein, 374 aa, aerococcus viridans
MNNNDIEYNAPSEIKYIDVCNTYDLEEEASKVVPHGGFNYIAGASGDEWTKRANDRAWKHKLLYPRLAQDVEAPDTSTEILGHKIKAPFIMAPIALHGLAHTTKEAGTARAVSEFGTIMSISAYSGATFEEISEGLNGGPRWFQIYMAKDDQQNRDILDEAKSDGATAIILTADSTVSGNRDRDCKNKFVYPFGMPIVQRYLRGTAEGMSLNNIYGASKQKISPRDIEEIAAHSGLPVFVKGIQHPEDADMAIKAGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY

SEQ ID NO: 21 - protein, 374 aa, aerococcus viridans
MNNNDIEYNAPSEIKYIDVCNTYDLEEEASKVVPHGGFNYIAGASGDEWTKRANDRAWKHKLLYPRLAQDVEAPDTSTEILGHKIKAPFIMAPIALHGLAHTTKEAGTARAVSEFGTIMSISAYSGATFEEISEGLNGGPRWFQIYMAKDDQQNRDILDEAKSDGATAIILTADSTVSGNRDRDCKNKFVYPFGMPIVQRYLRGTAEGMSLKNIYGASKQKISPRDIEEIAAHSGLPVFVKGIQHPEDADMAIKAGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY

SEQ ID NO: 22 - protein, 374 aa, aerococcus viridans
MNNNDIEYNAPSEIKYIDVVNTYDLEEEASKVVPHGGFNYIAGASGDEWTKRANDRAWKHKLLYPRLAQDVEAPDTSTEILGHKIKAPFIMAPIALHGLAHTTKEAGTARAVSEFGTIMSISAYSGATCEEISEGLNGGPRWFQIYMAKDDQQNRDILDEAKSCGATAIILTADSTVSGNRDRDVKNKFVYPFGMPIVQRYLRGTAEGMSLKNIYGASKQKISPRDIEEIAAHSGLPVFVKGIQHPEDADMAIKAGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY

SEQ ID NO: 23 - protein, 374 aa, aerococcus viridans
MNNNDIEYNAPSEIKYIDVCNTYDLEEEASKVVPHGGFNYIAGASGDEWTKRANDRAWKHKLLYPRLAQDVEAPDTSTEILGHKIKAPFIMAPIALHGLAHTTKEAGTARAVSEFGTIMSISAYSGATCEEISEGLNGGPRWFQIYMAKDDQQNRDILDEAKSCGATAIILTADSTVSGNRDRDCKNKFVYPFGMPIVQRYLRGTAEGMSLKNIYGASKQKISPRDIEEIAAHSGLPVFVKGIQHPEDADMAIKAGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY

SEQ ID NO: 24 - protein, 466 aa, Pichia pastoris
MNDDERKVTPEELAKHNTGTDCWVAINGKVYDLTEFLPQHPGGRNVILKRAGKDASKVFNPIHPPDAISKFLPADKFVGVLDGELPEEEEVLTNNNDIEYNAPSEIKYIDVVNTYDLEEEASKVVPHGGFNYIAGASGDEWTKRANDRAWKHKLLYPRLAQDVEAPDTSTEILGHKIKAPFIMAPISLHGLAHTTKEAGTARAVSEFGTIMSISAYSGATFEEISEGLNGGPRWFQIYMAKDDQQNRDILDEAKSDGATAIILTADSTVSGNRDRDVKNKFVYPFGMPIVQRYLRGTAEGMSLKNIYGASKQKISPRDIEEIAAHSGLPVFVKGIQHPEDADMAIKAGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY

SEQ ID NO: 25 - protein, 466 aa, Pichia pastoris
MNDDERKVTPEELAKHNTGTDCWVAINGKVYDLTEFLPQHPGGRNVILKRAGKDASKVFNPIHPPDAISKFLPADKFVGVLDGELPEEEEVLTNNNDIEYNAPSEIKYIDVCNTYDLEEEASKVVPHGGFNYIAGASGDEWTKRANDRAWKHKLLYPRLAQDVEAPDTSTEILGHKIKAPFIMAPIALHGLAHTTKEAGTARAVSEFGTIMSISAYSGATCEEISEGLNGGPRWFQIYMAKDDQQNRDILDEAKSCGATAIILTADSTVSGNRDRDCKNKFVYPFGMPIVQRYLRGTAEGMSLKNIYGASKQKISPRDIEEIAAHSGLPVFVKGIQHPEDADMAIKAGASGIWVSNHGARQLYEAPGSFDTLPAIAERVNKRVPIVFDSGVRRGEHVAKALASGADVVALGRPVLFGLALGGWQGAYSVLDYFQKDLTRVMQLTGSQNVEDLKGLDLFDNPYGYEY